(19) United States
(12) Patent Application Publication
(10) Pub. No.: US 2007/0191305 A1
(43) Pub. Date: Aug. 16, 2007

(54) Chromone complexes

(76) Inventors: Christophe Carola, Heidelberg (DE); Ralf Rosskopf, Muenster (DE); Herwig Buchholz, Frankfurt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/594,752

(22) PCT Filed: Mar. 7, 2005
(86) PCT No.: PCT/EP05/02369

§ 371 Date: Sep. 29, 2006

(30) Foreign Application Priority Data
Apr. 2, 2004 (DE) 10 2004 016 250.6

(51) Int. Cl. A61K 31/724; C08B 37/16

(52) U.S. Cl. 514/58; 536/103

(57) ABSTRACT

The invention relates to complexes of certain chromone derivatives, to compositions which comprise such derivatives, to corresponding processes for the preparation of the chromone derivatives or of compositions comprising same, and to the use thereof, in particular for the care, maintenance or improvement of the general state of the skin or hair.

The invention relates to complexes of certain chromone derivatives, to compositions which comprise such derivatives, to corresponding processes for the preparation of the chromone complexes or of the compositions comprising same, and to the use thereof, in particular for the care, maintenance or improvement of the general state of the skin or hair. In particular, the present invention also relates to cosmetic compositions for prophylaxis against ageing processes in the skin.

The human skin is subject to certain ageing processes, some of which are attributable to intrinsic processes (chronoageing) and some of which are attributable to exogenous factors (environmental, for example photoageing). In addition, temporary or even lasting changes to the skin picture can occur, such as acne, greasy or dry skin, keratoses, rosaceae, light-sensitive, inflammatory, erythematous, allergic or autoimmune-reactive reactions, such as dermatosis and photormatosis.

The exogenous factors include, in particular, sunlight or artificial radiation sources having a comparable spectrum, and compounds which can be formed by the radiation, such as undefined reactive photoproducts, which may also be free-radical or ionic. These factors also include cigarette smoke and the reactive compounds present therein, such as ozone, free radicals, for example the hydroxyl free radical, singlet oxygen and other reactive oxygen or nitrogen compounds which interfere with the natural physiology or morphology of the skin.

The influence of these factors can result, inter alia, in direct damage to the DNA of the skin cells and to the collagen, elastin or glycosaminoglycan molecules of the extracellular matrix, which are responsible for the strength of skin. In addition, the signal transduction chains, which are terminated by the activation of matrix-degrading enzymes, may be affected. Important representatives of these enzymes are the matrix metalloproteinases (MMPs, for example collagenases, gelatinases and stromelysins), whose activity is additionally regulated by TIMPs (tissue inhibitors of matrix metalloproteinases).

The consequences of the above-mentioned ageing processes are thinning of the skin, weaker interlacing of epidermis and dermis, and a reduction in the number of cells and the supplying blood vessels. This results in the formation of fine lines and wrinkles, the skin becomes leathery, and pigment defects can occur.

The same factors also act on hair, where damage can likewise occur. The hairs become brittle, less elastic and dull. The surface structure of the hairs is damaged.

Cosmetic or dermatological care products having properties which are claimed to counter the processes described or comparable processes or reduce or reverse the harmful consequences thereof are frequently distinguished by the following specific properties—free-radical-scavenging, anti-oxidative, inflammation-inhibiting or humectant. They prevent or reduce, inter alia, the activity of matrix-degrading enzymes or regulate the new synthesis of collagen, elastin or proteoglycans.

The use of antioxidants or free-radical scavengers in cosmetic compositions is adequately known per se. Thus, the use of the antioxidative vitamin E in sunscreen formulations is usual. Nevertheless, the effect achieved is even here well short of the hoped-for effect.

Vitamin A and vitamin A derivatives, such as retinoic acid, retinol and retinol esters, act on the differentiation of epithelial cells and are therefore employed for the prophylaxis and treatment of numerous phenomena which impair the skin state, for example use against acne, psoriasis, senile keratosis, skin discoloration and wrinkles has been described (cf., for example, WO 93/19743 and WO 02/02074).

However, a skin-irritant effect of retinol and derivatives is also described in the literature (for example WO 94/07462). These side effects restrict the use of retinol to narrowly limited areas, it being necessary to avoid over-dosing. There is therefore a demand for active ingredients which have a retinol-like spectrum of action, but do not have the side effects described or at least only do so in reduced form.

Owing to the constantly increasing demand for active ingredients for the preventative treatment of human skin and human hair against ageing processes and harmful environmental influences, the object of the present invention was to provide novel active ingredients which exhibit the effects already mentioned at the outset, are sufficiently oxidation- and photostable and can readily be formulated. The compositions prepared therewith should furthermore have as far as possible a low irritation potential for the skin, as far as possible have a positive influence on water binding in the skin, retain or increase skin elasticity and thus promote smoothing of the skin. In addition, they should preferably create a pleasant skin feeling on application to the skin.

The earlier German patent application DE 10337863.4 describes the use of at least one compound of the formula 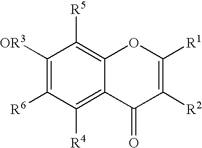
or a composition comprising at least one compound of this formula, where
R¹ and R² may be identical or different and are selected from
   H, —C(═O)—R⁷, —C(═O)—OR⁷,
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ- groups, where n=1 to 3,
R³ stands for H or straight-chain or branched C₁- to C₂₀-alkyl groups,
R⁴ stands for H or OR⁸,
R⁵ and R⁶ may be identical or different and are selected from
   —H and —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ stands for H, straight-chain or branched C₁- to C₂₀-alkyl groups, a polyhydroxyl compound, such as, preferably, an ascorbic acid radical or glycosidic radicals, and
R⁸ stands for H or straight-chain or branched C₁- to C₂₀-alkyl groups, where at least two of the substituents R¹, R², R⁴-R⁶ are other than H or at least one substituent from R¹ and R² stands for —C(═O)—R or —C(═O)—OR⁷, for the care, preservation or improvement of the general state of the skin or hair. On use of these compounds, there is a desire for administration forms which can be incorporated more easily into compositions, whose compositions exhibit increased storage stability or in which the bioavailability of the compounds is increased.

Surprisingly, it has now been found that complexing of these compounds with cyclodextrins results in products which meet the said requirements in an excellent manner.

The present invention therefore relates firstly to complex compounds of the formula I 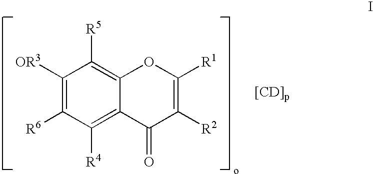
in which
R¹ and R² may be identical or different and are selected from
   H, —C(═O)—R⁷, —C(═O)—OR⁷,
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ- groups, where n=1 to 3,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ stands for H, straight-chain or branched C₁- to C₂₀-alkyl groups, a polyhydroxyl compound, such as, preferably, an ascorbic acid radical or glycosidic radicals, and
R⁸ stands for H or straight-chain or branched C₁- to C₂₀-alkyl groups, where at least 2 of the substituents R¹, R², R⁴—R⁶ are other than H or at least one substituent from R¹ and R² stands for —C(═O)—R⁷ or —C(═O)—OR⁷,
CD stands for a cyclodextrin molecule
o stands for the number 1 and
p stands for a number from the range 0.5 to 3.

The present invention relates secondly to compositions comprising a suitable vehicle, characterised in that the compositions comprises
0.005 to 99% by weight of a complex compound of the formula I containing radicals as described above, or the composition comprises
0.002 to 70% by weight of cyclodextrin and
0.001 to 60% by weight of at least one compound of the formula II or topically tolerated salts and/or derivatives thereof 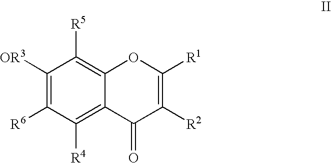
   where
R¹ and R² may be identical or different and are selected from
   H, —C(═O)—R⁷,—C(═O)—OR⁷,
   straight-chain or branched C₁- to C₂₀-alkyl groups,
   straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ- groups, where n=1 to 3,
R³ stands for H or straight-chain or branched C₁- to C₂₀-alkyl groups,
R⁴ stands for H or OR⁸,
R⁵ and R⁶ may be identical or different and are selected from
   —H, —OH,
   straight-chain or branched C₁- to C₂₀-alkyl groups,
   straight-chain or branched C₃- to C₂₀-alkenyl groups,
   straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ stands for H, straight-chain or branched C₁- to C₂₀-alkyl groups, a polyhydroxyl compound, such as, preferably, an ascorbic acid radical or glycosidic radicals, and
R⁸ stands for H or straight-chain or branched C₁- to C₂₀-alkyl groups, where at least 2 of the substituents R¹, R², R⁴—R⁶ are other than H or at least one substituent from R¹ and R² stands for —C(═O)—R⁷ or —C(═O)—OR⁷.

For the purposes of the present invention, the expression “compound of the formula I or II” basically also encompasses the salts of the respective compounds of the formula I and II. The preferred salts here include, in particular, alkali metal and alkaline earth metal salts as well as ammonium salts, but in particular sodium and potassium salts.

The compositions according to the invention here are usually either compositions which can be used topically, for example cosmetic or dermatological formulations, or medicaments or foods or food supplements. The compositions comprise a cosmetically or dermatologically or pharmaceutically or food-suitable excipient and, depending on the desired property profile, optionally further suitable ingredients.

Cyclodextrins are built up from 6, 7, 8 or even more α-1,4-linked glucose units, with cycloohexaamylose (alpha- or x-cyclodextrin) being distinguished by the structure

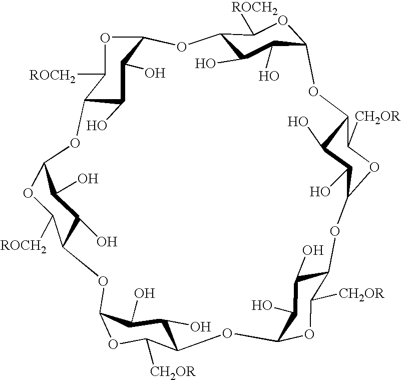

Cycloheptaamylose (beta- or β where bonded to this glycoside radical, in each case via an —O— group, is at least one radical selected from β-cyclodextrin) is distinguished by the structure

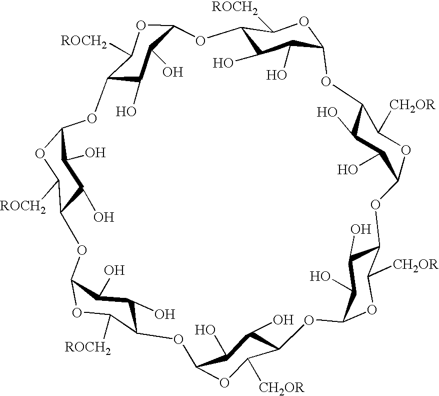

Cyclooctaamylose (gamma- or γ-cyclodextrin) is distinguished by the structure

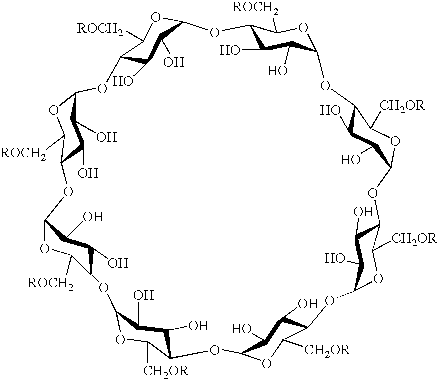

Cycloenneaamylose (delta- or δ-cyclodextrin) is distinguished by the structure

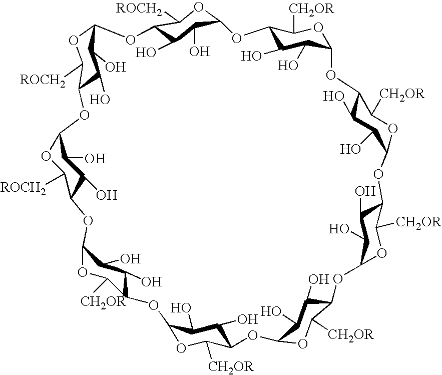

Cyclodextrins may occur in underivatised form (R=H) or also in derivatised form, for example alkoxylated, hydroxyalkylated or alkylated, in particular propoxylated or methylated, in position R.

Chromone-cyclodextrin complexes are kown in principle:
M. Christoff, L. T. Okano, C. Bohne, J. Photochem. and Photobiol, A: Chemistry, 134 (2000) pp. 169-176, are concerned with flavone and chromone-β-cyclodextrin complexes. The dynamics od the complex formation is investigated with reference to the parent flavone and chromone compounds.
M. Milewski, W. Urjasz, A. Maciejewski, W. Augustyniak, Polish J. Chem. 72 (1998) pp. 2405-2417, investigate various β-cyclodextrin complexes of aromatic ketones. It is found that 1:1 β-cyclodextrin complexes of chromone or 2-butylchromone form.

Applications of various chromone derivatives are likewise known from the literature:

The use of certain 2-(alkyl)carboxyl- or 2-(alkyl)phenyl-substituted chromen-4-one derivatives in combination with divalent zinc in pharmaceutical and cosmetic compositions is disclosed in EP-A-0 304 802. The compositions are suitable for the treatment of skin, in particular for the treatment of dermatoses, including atopic eczema. EP-A-0 424 444 discloses the use of salts of chromonecarboxylic acid in cosmetics for combating skin ageing. The compound exhibits a UV-filtering action here and has the following effects in animal experiments: the proportion of bound lipids in the skin increases, the proportion of soluble collagen in the skin is increased, the resistance of the skin to the effects of the fibroplatic proteases collagenase and elastase is increased.

U.S. Pat. No. 6,019,992 discloses cosmetic compositions which comprise 4-chromanone and are suitable for the treatment of aged, dry or wrinkled skin. It is shown here that 4-chromanone promotes cell differentiation and stimulates lipid production in keratinocyte cultures.

EP-A-1 216 692 discloses the use of 2-methyl-2-(β-carboxyethyl)chroman derivatives in cosmetic compositions. The said compositions are particularly suitable for prophylaxis against ageing processes of skin and hair and for prophylaxis against dry skin, wrinkle formation and pigment defects.

Compositions for topical application which comprise chromone derivatives, such as, for example, chromone, 7-hydroxychromone, 7-methoxychromone, 5,7-dihydroxy-2-methylchromone, 3-methyl-2-butenyloxychromone, 3-acetyl-5,7-dihydroxy-2-methylchromone, 5-hydroxychromone, n-pentyl 7-methoxychromone-2-carboxylate, n-undecyl 5-methoxychromone-2-carboxylate, 5-hydroxy-7-methoxy-2-methylchromone, 7-methoxychromone-2-carboxylic acid, n-pentylchromone-2-carboxylic acid, 5-methoxychromone and chromone-2-carboxylic acid, are disclosed in Japanese patent application JP 05/301813. The chromone derivatives act as skin-tolerated tyrosinase inhibitors which reduce hyperpigmentation of the skin.

Japanese patent application JP 09/188608 discloses the use of substituted chromone derivatives, such as, in particular, 5,7-dihydroxychromones, 7-methoxychromones, 5-hydroxy-7-methoxy-2-methylchromone and 5-hydroxy-2-methylchromone, as active ingredient against grey hair. The action here is attributed to activation of the coloured pigment-forming cells and the increase in melanogenesis.

A composition against skin ageing comprising chromone derivatives which are substituted in the 2-position by C₁₋₁₅-alkyl and have H, OH or alkoxy substitution in the 7-position, in combination with aminopropanol derivatives is disclosed in JP 10/194919.

Cosmetic compositions which comprise substituted chromone derivatives, such as, for example, 2-(1-ethylpentyl)chromone, 5,7-dihydroxychromones, 7-methoxychromones, 5-hydroxy-7-methoxy-2-methylchromone and 5-hydroxy-2-methylchromone, and aromatic compounds having a melting point of −10° C. or above are disclosed in JP 10/114640. The chromone derivative here simplifies incorporation of the aromatic compound into the cosmetic formulation.

It has been found, in an unforeseeable manner for the person skilled in the art, that complex compounds of the formula I or compositions for topical use comprising the above-mentioned complex compounds of the formula I or compounds of the formula II and cyclodextrins remedy the disadvantages of the prior art.

It is particularly advantageous here if the cyclodextrins used are γ-cyclodextrins, preferably gamma-cyclodextrins which are substituted by C₁₋₂₄-alkyl or C₁₋₂₄-hydroxyalkyl on one or more hydroxyl groups, such as, in particular, hydroxypropyl-y-cyclodextrin, or mixtures of cyclodextrins which comprise at least 30% by weight, based on the total weight of the cyclodextrin mixture, of the above-mentioned γ-cyclodextrins.

It is furthermore advantageous for the content of cyclodextrins to be 0.01-20.0% by weight, preferably 0.05-10.0% by weight, particularly preferably 0,1-5.0% by weight, in each case based on the total weight of the composition. The proportion of the compounds of the formula II in the composition here is preferably 0.01 to 20% by weight, particularly preferably 0.05 to 10% by weight and especially preferably 0.1 to 5% by weight, based on the composition as a whole. The proportion of the compounds of the formula II in the composition is very particularly preferably 0.1 to 2% by weight, based on the composition as a whole.

The active-ingredient combinations in accordance with the invention or cosmetic or dermatological compositions comprising such active-ingredient combinations are satisfactory preparations in every respect. It was not foreseeable for the person skilled in the art that the compositions in accordance with the invention
provide compounds of the formula II in increased bioavailability,
maintain or restore the barrier properties of the skin better,
counter drying-out of the skin better and
protect the skin against environmental influences better than the compositions of the prior art.

Uses preferred in accordance with the invention of the compounds of the formula I or of compositions comprising at least one compound of the formula I here are, in particular, the use for prophylaxis against time- and/or light-induced ageing processes of the human skin or human hair, in particular for prophylaxis against dry skin, wrinkling and/or pigment defects, and/or for reducing or preventing damaging effects of UV rays on the skin, and for prophylaxis against or reduction of skin unevenness, such as wrinkles, fine lines, rough skin or large-pored skin.

Preferred uses in accordance with the invention of the compounds of the formula I or of compositions comprising at least one compound of the formula I are furthermore the use for the prevention of premature skin ageing, in particular for the prophylaxis and/or prevention of light- or ageing-induced wrinkling of the skin, for the prevention of pigmentation and keratosis actinica, and for the prophylaxis and/or treatment of skin diseases associated with a defect in keratinisation which affects differentiation and cell proliferation, in particular for the treatment of acne vulgaris, acne comedonica, polymorphic acne, acne rosaceae, nodular acne, acne conglobata, age-induced acne, acne which arises as a side effect, such as acne solaris, medicament-induced acne or acne professionalis, for the treatment of other defects in keratinisation, in particular ichthyosis, ichthyosiform states, Darier's disease, keratosis palmoplantaris, leukoplakia, leukoplakiform states, herpes of the skin and mucous membrane (buccal) (lichen), for the treatment of other skin diseases associated with a defect in keratinisation and which have an inflammatory and/or immunoallergic component and in particular all forms of psoriasis which affect the skin, mucous membranes and fingers and toenails, and psoriatic rheumatism and skin atopy, such as eczema or respiratory atopy, or hypertrophy of the gums, and for the prophylaxis and/or treatment of all benign or malignant excrescence of the dermis or epidermis, which may be of viral origin, such as verruca vulgaris. verruca plana, epidermodysplasia verruciformis, oral papillomatosis, papillomatosis florida, and excrescence which may be caused by UV radiation, in particular epithelioma baso-cellulare and epithelioma spinocellulare.

It is assumed that the preferred compounds of the formula I also act as enzyme inhibitors. They are thought to inhibit histidine decarboxylase, protein kinases, elastase, aldose reductase and hyaluronidase, and therefore enable the intactness of the basic substance of vascular sheaths to be maintained. Furthermore, they presumably inhibit non-specifically catechol O-methyl transferase, causing the amount of available catecholamines and thus the vascular strength to be increased. Furthermore, they inhibit AMP phosphodiesterase, giving the substances potential for inhibiting thrombocyte aggregation.

Owing to these properties, the compositions according to the invention are, in general, suitable for immune protection and for the protection of DNA and RNA. In particular, the compositions are suitable for the protection of DNA and RNA against oxidative attack, against free radicals and against damage due to radiation, in particular UV radiation. A further advantage of the compositions according to the invention is cell protection, in particular protection of Langerhans cells against damage due to the above-mentioned influences. All these uses and the use of the compounds of the formula I for the preparation of compositions which can be employed correspondingly are expressly also a subject-matter of the present invention.

The invention also relates here in each case to the use of the compounds of the formula I for the preparation of compositions suitable for the above-mentioned uses.

On use of the complex compounds used in accordance with the invention or cosmetic or topical dermatological compositions having an active content of active-ingredient combinations used in accordance with the invention, effective treatment, but also prophylaxis,
of deficient, sensitive or hypoactive skin states or deficient, sensitive or hypoactive states of skin appendages,
of adverse changes in the skin and skin appendages caused by the environment (smoke, smog, reactive oxygen species, free radicals) and in particular light,
of skin damage caused by light,
of pruritus,
of dry skin states and horny layer barrier defects,
of inflammatory skin states and atopic eczema, seborrhoeic eczema, polymorphic light dermatosis, psoriasis, vitiligo,
   is surprisingly possible.

It is also in accordance with the invention to use the complex compounds of the formula I or the compositions comprising the compounds of the formula II and cyclodextrins
for the cosmetic or dermatological treatment or prophylaxis of undesired skin states,
for the prophylaxis and treatment of inflammatory skin states—also atopic eczema,
for skin protection in the case of dry skin determined to be sensitive,
for the protection of the skin against photoreactions,
for the treatment and prophylaxis of sensitive skin states.

The complex compounds or compositions comprising the active-ingredient combination in accordance with the invention have a synergistic action in relation to the individual components in all these uses.

Advantageous in accordance with the invention is the use of cyclodextrins and/or cyclodextrin derivatives for increasing the solubility of compounds of the formula II. Furthermore advantageous is the use of cyclodextrins and/or cyclodextrin derivatives for improving the biological efficacy of compounds of the formula II.

The use according to the invention of chromen-4-one derivatives of the general formula I in compositions offers, inter alia, protection against damage caused directly or indirectly by UV radiation or by processes caused by reactive compounds, such as, for example, skin ageing, loss of skin moisture, loss of skin elasticity, formation of wrinkles or lines or of pigment defects or age spots.

The present invention furthermore relates to the use of the above-mentioned compositions for the prevention of undesired changes in the skin picture, such as, for example, acne or greasy skin, keratoses, light-sensitive, inflammatory, erythematous, allergic or autoimmune-reactive reactions.

However, the compounds and compositions according to the invention preferably also serve for calming sensitive and irritated skin, for the preventative regulation of collagen, hyaluronic acid and elastin synthesis, stimulation of DNA synthesis, in particular in the case of deficient or hypoactive skin states, regulation of the transcription and translation of matrix-degrading enzymes, in particular of MMPs, increasing cell regeneration and regeneration of the skin, increasing the skin's own protective and repair mechanisms for DNA, lipids and/or proteins.

Preferred compounds of the formula I are characterised in that R³ stands for H and R⁴ stands for OH, since the action potential of representatives of this class of compound is particularly high in the above-mentioned sense. If, in addition, at least one of the radicals R⁵ and R⁶ stands for OH, these preferred compounds, in addition to the above-mentioned properties, additionally have an antioxidant potential. They can therefore simultaneously function as antioxidant in compositions.

Other preferred compounds of the formula I are characterised in that R⁵ and R⁶ stand for H. In this case, the radicals R³ and R⁴ are freely accessible, which, as assumed, is advantageous for interaction with enzymes involved in the effects mentioned.

Likewise preferred compounds of the formula I are characterised in that one of the radicals R¹ and R² stands for H and the other radical stands for —C(═O)—R⁷, —C(═O)—OR⁷ or a straight-chain or branched C₁- to C₂₀-alkyl group.

Glycosidic radicals which can be employed are in particular mono- or oligosaccharide radicals. Preference is given here to hexosyl radicals, in particular ramnosyl radicals and glucosyl radicals. However, other hexosyl radicals, for example allosyl, altrosyl, galactosyl, gulosyl, idosyl, mannosyl and talosyl, may also advantageously be used. It may also be advantageous to use pentosyl radicals. The glycosyl radicals may be linked to the basic structure by means of an α- or β-glycosidic link. A preferred disaccharide is, for example, 6-O-(6-deoxy-α-L-mannopyranosyl)-β-D-glucopyranoside.

The chromone moiety of compound I is preferably a compound selected from the compounds of the formulae IIa-IIn:

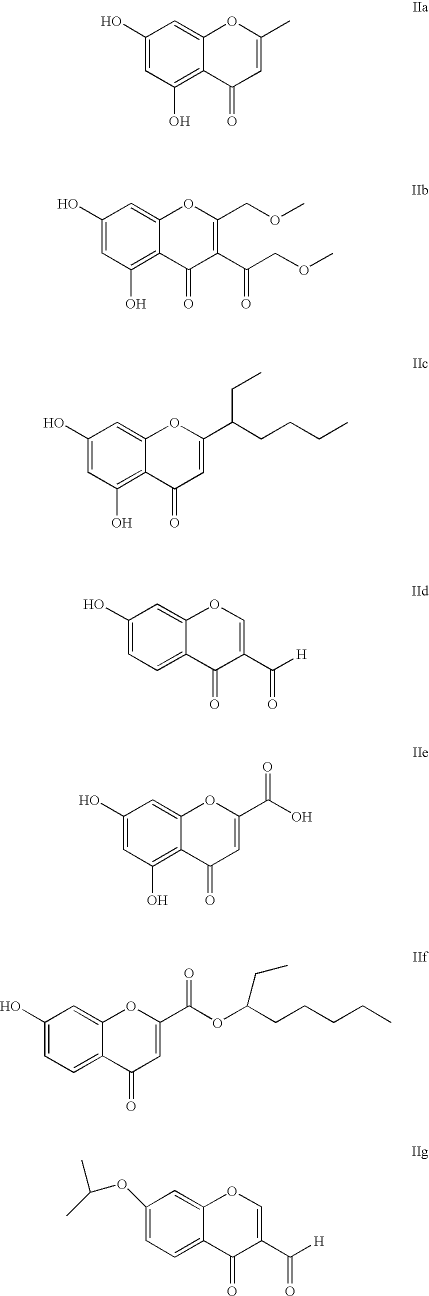

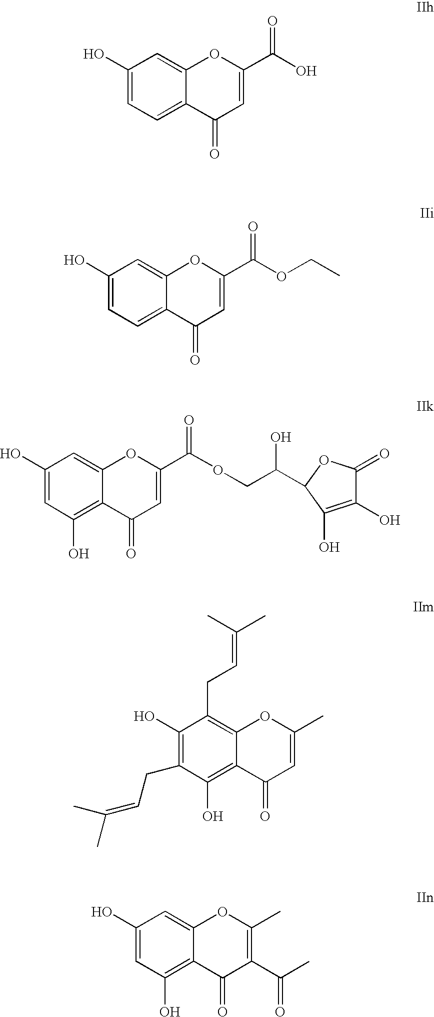

The chromone moieties of the compounds of the formula I or compounds of the formula II can be isolated or prepared by methods which are well known to the person skilled in the art and are described in the literature (for example in standard works, such as Houben-Weyl, Methodn der organischen Chemie [Methods of Organic Chemistry], Georg-Thieme-Verlag, Stuttgart).

For example, 5,7-dihydroxy-2-methylchromen-4-one occurs in plants and can be isolated by extraction. The plant extracts are prepared by conventional methods of extraction from the plants or plant parts. Suitable extraction methods may be: maceration, remaceration, digestion, agitation maceration, fluidised-bed extraction, ultrasound extraction, countercurrent extraction, percolation, repercolation, evacolation, diacolation or solid/liquid extraction with continuous reflux, which is carried out in a Soxhlet extractor.

The solvent used for the extraction can be, for example, water or an alcohol.

It can be ascribed to the general knowledge of the person skilled in the art how these extractions can be carried out in detail and the resultant crude extracts can be purified by generally conventional methods.

One possible synthetic route for 5,7-dihydroxy-2-methylchromen-4-one is, for example, also described in B. Vermes, H. Wagner, Stud. Org. Chem. (Amsterdam) (1982), Volume date 1981, 11 (Flavonoids, Bioflavonoids), 161-167 and in B. Vermes, V. M. Chari, H. Wagner, Helv. Chim. Acta (1981), 64(4), 1964-1967.

The synthesis of 5,7-dihydroxy-2-methylchromen-4-one is shown in scheme 1. 4′,7-Dibenzylkaempferol (1) [H. Wagner, H. Danninger, O. Seligmann, M. Nógrádi, L. Farkas, N. Farnsworth, Chem. Ber. 103 (1978) 3768] is reacted with 2,3,4-tri-O-acetyl-6-O-chloroacetyl-β-D-glucopyranosyl bromide (2) in the presence of Ag₂CO₃ and pyridine to give compound 3. Compound 2can be prepared by the method described in D. Y. Gagniere, P. J. A. Wottero, Carbohydrate Res. 28 (1973) 1965. Catalytic debenzylation and subsequent careful acetylation of compound 3 gives compound 4, from which compound 5 can be obtained after removal of the chloroacetyl group using thiourea. In this compound, only one hydroxyl group is free, meaning that the esterification of compound 5 can proceed selectively. The esterification using the acid chloride p-acetylcoumaroyl chloride 6 can be carried out in a mixture of pyridine and dichloromethane. An excess of acid chloride and a long reaction time (about 96 h) at room temperature is necessary to ensure that the esterification proceeds to completion. The final step, the selective saponification of the 7 acetyl groups in compound 7, can be carried out by the method described in G. Zemplen, Chem. Ber. 59 (1926) 1258. This is carried out using a catalytic amount of NaOCH₃ and a calculated amount of methanol.

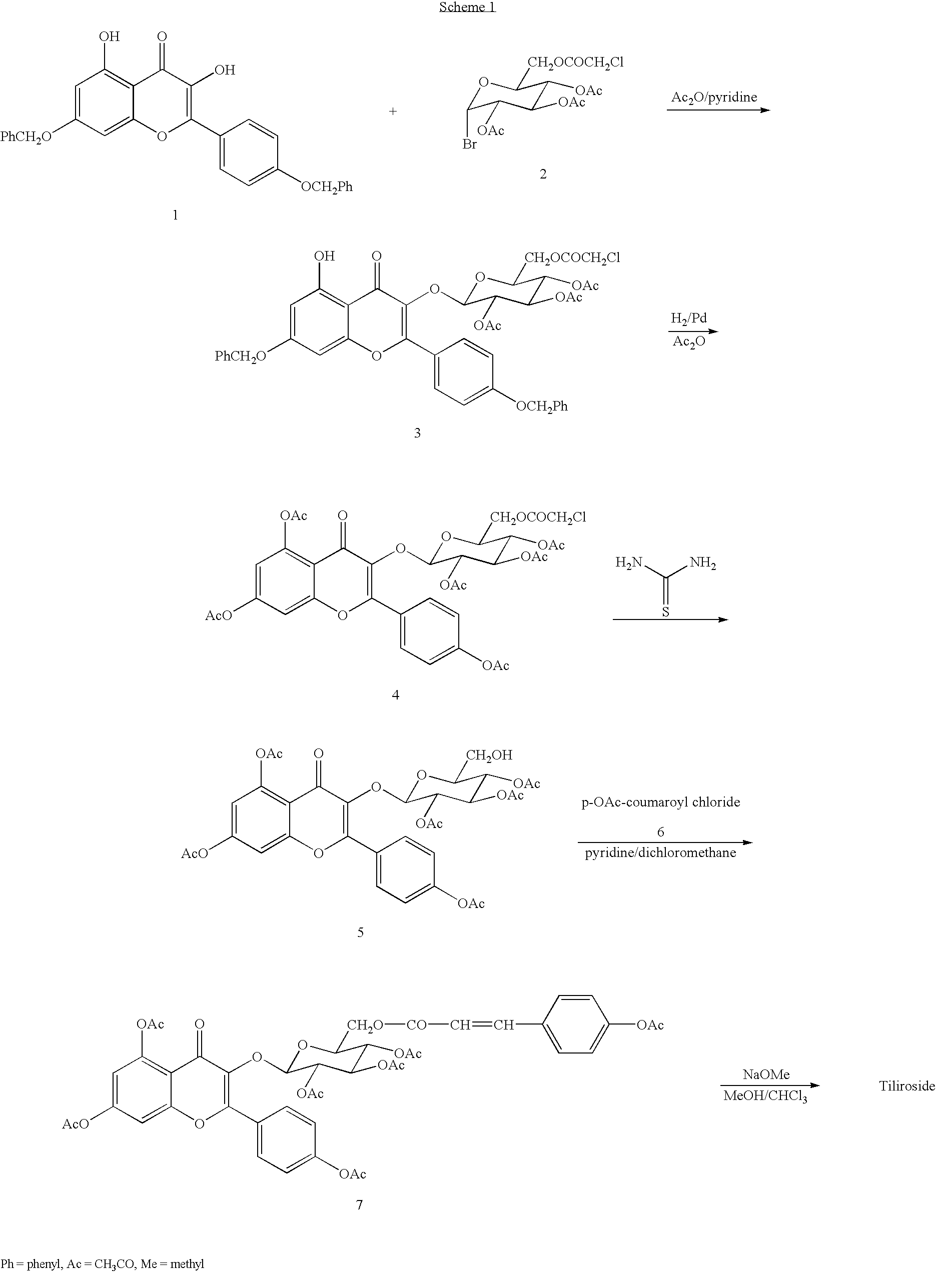

Other chromone moieties of the compounds of the formula I or compounds of the formula II can be obtained by routine modification of the synthesis shown in scheme 1. Depending on the target molecule, different starting materials are used here, i.e. other optionally protected chromones, sugar components and radicals which are to be attached to the sugar component.

The esterification of glycosidic OH groups using aromatic sulfonic acid units can be carried out, for example, by the method described in A. B. Foster et al., J. Chem. Soc. (1954) 3625-3629. According to this method, the sugar component can, for example, be reacted with a corresponding aromatic sulfonyl chloride in pyridine.

The etherification of glycosidic OH groups using aromatic radicals can be carried out, for example, by the method described in P. Beraud et al., Tetrahedron Let. 30(3) (1989) 325-326. In this Mitsunobu reaction, the etherification is carried out, for example, by dissolving the sugar component in pyridine together with triphenylphosphine PPh₃ and reacting the solution with a corresponding phenol component and diethyl azodicarboxylate.

The etherification of glycosidic OH groups using radicals of saturated hydrocarbons can be carried out, for example, by the method described in M. Goebel et al., Tetrahedron 53(9) (1997) 3123-3134. The etherification is carried out, for example, by carefully adding sodium hydride to the sugar component in dry dimethylformamide under inert gas and then carefully reacting the mixture with a suitable alkylating reagent, such as, for example, a corresponding bromide.

The complex compounds of the formula I can be prepared by reacting compounds of the formula II with cyclodextrins in solution, preferably at elevated temperature. The present invention furthermore relates to a corresponding process.

It has been found that complexes comprising about 2 mol of cyclodextrin per mole of chromone of the formula II meet the requirements according to the invention in a particular manner. It is therefore preferred in accordance with the invention for o in formula I to be equal to 1 and p to be in the range from 1.75 to 2.1, preferably for p to be equal to 2.

Corresponding compounds can be prepared if the cyclodextrin is employed in excess or precisely in the molar ratio 2:1, based on the chromone.

In a preferred embodiment of the present invention, the composition is a composition for the protection of body cells against oxidative stress, in particular for reducing skin ageing, characterised in that it comprises one or more further antioxidants besides the one or more compounds of the formula I or of the formula II.

There are many proven substances known from the specialist literature which can be used as antioxidants, for example amino acids (for example glycine, histidine, tyrosine, tryptophan) and derivatives thereof, imidazoles, (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and hepta-thionine sulfoximine) in very low tolerated doses (for example pmol to μmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic compositions according to the invention. Known and commercial mixtures are, for example, mixtures comprising, as active ingredients, lecithin, L-(+)-ascorbyl palmitate and citric acid (for example (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-x-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with compounds of the formula I or formula II in such compositions in ratios in the range from 1000:1 to 1:1000, preferably in amounts of 100:1 to 1:100.

The compositions according to the invention may comprise vitamins as further ingredients. The cosmetic compositions according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I or formula II in ratios in the range from 1000:1 to 1:1000, preferably in amounts of 100:1 to 1:100.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology & Medicine 2001, 31(7), 869-881, are investigating the pH dependence of the antioxidant action of hydroxyflavones. Quercetin exhibits the greatest activity amongst the structures investigated over the entire pH range.

Suitable antioxidants are furthermore compounds of the formula III 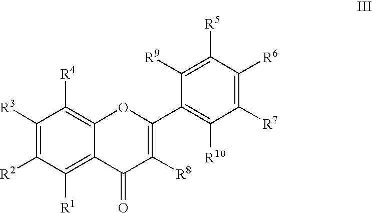
where R¹ to R¹⁰ may be identical or different and are selected from
H
OR¹¹
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ- groups, where n=1 to 3,
where all OR¹¹, independently of one another, stand for
   OH
   straight-chain or branched C₁- to C₂₀-alkoxy groups,
   straight-chain or branched C₃- to C₂₀-alkenyloxy groups,
   straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
   C₃- to C₁₀-cycloalkoxy groups and/or C₃- to C₁₂-cycloalkenyloxy groups, where the rings may each also be bridged by —(CH₂)ₙ- groups, where n=1 to 3, and/or
   mono- and/or oligoglycosyl radicals,
with the proviso that at least 4 radicals from R¹ to R⁷ stand for OH and that at least 2 pairs of adjacent —OH groups are present in the molecule,
or R^{2,} R⁵ and R⁶ stand for OH and the radicals R¹, R^{3,} R⁴ and R⁷⁻¹⁰ stand for H,
   as described in German patent application DE-A 10244282.

Compositions which are particularly preferred in accordance with the invention also comprise UV filters in addition to the compounds of the formula I or formula II.

On use of the dibenzoylmethane derivatives which are particularly preferred as UV-A filters in combination with the compounds of the formula I or formula II, an additional advantage arises: the UV-sensitive dibenzoylmethane derivatives are additionally stabilised by the presence of the compounds of the formula I or formula II. The present invention therefore furthermore relates to the use of the compounds of the formula I or formula II for the stabilisation of dibenzoylmethane derivatives in compositions.

In principle, all UV filters are suitable for combination with the compounds of the formula I or formula II according to the invention. Particular preference is given to UV filters whose physiological acceptability has already been demonstrated. Both for UVA and UVB filters, there are many proven substances known from the specialist literature, for example
benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-[(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL),
benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020),
benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),
methoxycinnamic acid esters, such as octyl methoxycinnamate (for example Eusolex® 2292), isopentyl 4-methoxycinnamate, for example as a mixture of the isomers (for example Neo Heliopan® E 1000),
salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS),
4-aminobenzoic acid and derivatives, such as 4-aminobenzoic acid, 2-ethylhexyl 4-(dimethylamino)benzoate (for example Eusolex® 6007), ethoxylated ethyl 4-aminobenzoate (for example Uvinul®) P25),
phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;
   and further substances, such as
2-ethylhexyl 2-cyano-3,3-diphenylacrylate (for example Eusolex® OCR),
3.3′-(1,4-phenylenedimethylene)bis(7,7-dimethyl-2-oxobicyclo[2.2.1]hept-1-ylmethanesulfonic acid and salts thereof (for example Mexoryl® SX) and
2,4,6-trianilino-(p-carbo-2′-ethylhexyl-1′-oxy)-1,3,5-triazine (for example Uvinul® T 150)
hexyl 2-(4-diethylamino-2-hydroxybenzoyl)benzoate (for example Uvinul®UVA Plus, BASF).

The compounds mentioned in the list should only be regarded as examples. It is of course also possible to use other UV filters.

These organic UV filters are generally incorporated into cosmetic formulations in an amount of 0.5 to 10 per cent by weight, preferably 1-8%.

Further suitable organic UV filters are, for example,
2-(2H-benzotriazol-2-yl)-4-methyl-6-(2-methyl-3-(1,3,3,3-tetramethyl-1 -(trimethylsilyloxy)disiloxanyl)propyl)phenol (for example Silatrizole®)),
2-ethylhexyl 4.4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and approximately 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl])phenoxy)propenyl) and 0.1 to 0.4% of (methylhydrogen]silylene]] (n≈60) (CAS No. 207 574-74-1)
2.2′-methylenebis(6-(2H-benzotriazol-2-yl)-4-(1,1,3,3-tetramethylbutyl)phenol) (CAS No. 103 597-45-1)
2.2′-(1,4-phenylene)bis(1H-benzimidazole-4,6-disulfonic acid, monosodium salt) (CAS No. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).
2-ethylhexyl 4.4′-[(6-[4-((1,1-dimethylethyl)aminocarbonyl)phenylamino]-1,3,5-triazine-2,4-diyl)diimino]bis(benzoate) (for example Uvasorb® HEB),

Further suitable UV filters are also methoxyflavones corresponding to the earlier German patent application DE-A 10232595.

Organic UV filters are generally incorporated into cosmetic formulations in an amount of 0.5 to 20 per cent by weight, preferably 1-15%.

Conceivable inorganic UV filters are those from the group of the titanium dioxides, such as, for example, coated titanium dioxide (for example Eusolex® T-2000, Eusolex® T-AQUA, Eusolex® T-AVO), zinc oxides (for example Sachtotec®), iron oxides or also cerium oxides. These inorganic UV filters are generally incorporated into cosmetic compositions in an amount of 0.5 to 20 per cent by weight, preferably 2-10%.

Preferred compounds having UV-filtering properties are 3-(4′-methylben-zylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and potassium, sodium and triethanolamine salts thereof.

The protective action against damaging effects of UV radiation can be optimised by combining one or more compounds of the formula I or formula II with further UV filters.

Optimised compositions may comprise, for example, the combination of the organic UV filters 4′-methoxy-6-hydroxyflavone with 1-(4-tert-butyl-phenyl)-3-(4-methoxyphenyl)propane-1,3-dione and 3-(4′-methylbenzyli-dene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous compositions. In addition, the oily impression on application of the composition comprising hydrophobic UV filters, which is frequently regarded as unpleasant, is suppressed.
Certain UV filters, in particular dibenzoylmethane derivatives, exhibit only reduced photostability in cosmetic compositions. Encapsulation of these filters or compounds which impair the photostability of these filters, such as, for example, cinnamic acid derivatives, enables the photostability of the entire composition to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables composition problems caused by the interaction of individual composition constituents with one another, such as crystallisation processes, precipitation and agglomerate formation, to be avoided since the interaction is suppressed.

It is therefore preferred in accordance with the invention for one or more of the above-mentioned UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by a sol-gel process, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in compositions according to the invention are preferably present in amounts which ensure that the encapsulated UV filters are present in the composition in the above-indicated amounts.

The compositions according to the invention may in addition comprise further conventional skin-protecting or skin-care active ingredients. These may in principle be any active ingredients known to the person skilled in the art.

It may furthermore be preferred for the composition according to the invention to comprise at least one repellent, where the repellent is preferably selected from N,N-diethyl-3-methylbenzamide, ethyl 3-(acetylbutylamino)propionate, dimethyl phthalate, butopyronoxyl, 2,3,4,5-bis(2-butylene)tetrahydro-2-furaldehyde, N,N-diethylcaprylamide, N,N-diethylbenzamide, o-chloro-N,N-diethylbenzamide, dimethyl carbate, di-n-propyl isocinchomeronate, 2-ethylhexane-1,3-diol, N-octylbicycloheptenedicarboximide, piperonyl butoxide, 1-(2-methylpropoxycarbonyl)-2-(hydroxyethyl)piperidine, or mixtures thereof, where it is particularly preferably selected from N,N-diethyl-3-methylbenzamide, ethyl 3-(acetylbutylamino)propionate 1-(2-methylpropoxycarbonyl)-2-(hydroxyethyl)piperidine, or mixtures thereof.

The compositions according to the invention which comprise repellents are preferably insect repellents. Insect repellents are available in the form of solutions, gels, sticks, rollers, pump sprays and aerosol sprays, with solutions and sprays forming the majority of the commercially available products. The basis for these two product forms is usually formed by alcoholic or aqueous/alcoholic solutions with addition of fatting substances and slight perfuming.

Particularly preferred active ingredients are, for example, also so-called compatible solutes. These are substances which are involved in the osmoregulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and the respective precursors thereof. For the purposes of German patent application DE-A-10133202, osmolytes are taken to mean, in particular, substances from the group of the polyols, such as, for example, myo-inositol, mannitol or sorbitol and/or one or more of the osmolytically active substances mentioned below:
taurine, choline, betaine, phosphorylcholine, glycerophosphorylcholines, glutamine, glycine, o-alanine, glutamate, aspartate, proline, and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

In accordance with the invention, compatible solutes are preferably substances selected from the group consisting of pyrimidinecarboxylic acids (such as ectoine and hydroxyectoine), proline, betaine, glutamine, cyclic diphosphoglycerate, N-acetylornithine, trimethylamine N-oxide, di-myoinositol phosphate (DIP), cyclic 2,3-diphosphoglycerate (cDPG), 1,1- diglycerol phosphate (DGP), β-mannosyl glycerate (firoin), β-mannosylglyceramide (firoin A) or/und dimannosyl diinositol phosphate (DMIP) or an optical isomer, derivative, for example an acid, or a salt or ester of these compounds, or combinations thereof.

Of the pyrimidinecarboxylic acids, particular mention should be made here of ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoine and ectoine derivatives, such as hydroxyectoine, can advantageously be used in medicaments. In particular, hydroxyectoine can be employed for the preparation of a medicament for the treatment of skin diseases. Other areas of application of hydroxyectoine and other ectoine derivatives are typically in areas in which, for example, trehalose is used as additive. Thus, ectoine derivatives, such as hydroxyectoine, can be used as protectant in dried yeast and bacteria cells. Pharmaceutical products, such as non-glycosylated, pharmaceutically active peptides and proteins, for example t-PA, can also be protected with ectoine or its derivatives.

Of the cosmetic applications, particular mention should be made of the use of ectoine and ectoine derivatives for the care of aged, dry or irritated skin. Thus, European patent application EP-A-0 671 161 describes, in particular, that ectoine and hydroxyectoine are employed in cosmetic compositions, such as powders, soaps, surfactant-containing cleansing products, lipsticks, rouge, make-ups, care creams and sunscreen compositions.

Preference is given here to the use of a pyrimidinecarboxylic acid of the following formula IV 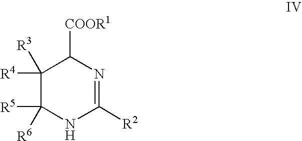
in which R¹ is a radical H or C1-8-alkyl, R² is a radical H or C1-4-alkyl, and R³, R⁴, R⁵ and R⁶ are each, independently of one another, a radical from the group H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoine ((S)-1,4,5,6-tetrahydro-2-methyl-4-pyrimidinecarboxylic acid) and hydroxyectoine ((S,S)-1,4,5,6-tetrahydro-5-hydroxy-2-methyl-4-pyrimidinecarboxylic acid). The compositions according to the invention preferably comprise pyrimidinecarboxylic acids of this type in amounts of up to 15% by weight. The pyrimidinecarboxylic acids are preferably employed here in ratios of from 100:1 to 1:100 with respect to the compounds of the formula I, with ratios in the range from 1:10 to 10:1 being particularly preferred.

It is particularly preferred in accordance with the invention if the compatible solutes are selected from di-myo-inositol phosphate (DIP), cyclic 2,3-diphosphoglycerate (cDPG), 1,1- diglycerol phosphate (DGP), β-mannosyl glycerate (firoin), β- mannosylglyceramide (firoin-A) or/and di-mannosyl diinositol phosphate (DMIP), ectoine, hydroxyectoine or mixtures thereof.

Of the aryl oximes that are likewise preferably employed, preference is given to the use of 2-hydroxy-5-methyllaurophenone oxime, which is also known as HMLO, LPO or F5. Its suitability for use in cosmetic compositions is disclosed, for example, in DE-A-41 16 123. Compositions which comprise 2-hydroxy-5-methyllaurophenone oxime are accordingly suitable for the treatment of skin diseases which are accompanied by inflammation. It is known that compositions of this type can be used, for example, for the therapy of psoriasis, various forms of eczema, irritative and toxic dermatitis, UV dermatitis and further allergic and/or inflammatory diseases of the skin and integumentary appendages. Compositions according to the invention which, in addition to the compound of the formula I, additionally comprise an aryl oxime, preferably 2-hydroxy-5-methyllaurophenone oxime, exhibit surprising antiinflammatory suitability. The compositions here preferably comprise 0.01 to 10% by weight of the aryl oxime, it being particularly preferred for the composition to comprise 0.05 to 5% by weight of aryl oxime.

In a further, likewise preferred embodiment of the present invention, the composition according to the invention comprises at least one self-tanning agent.

Advantageous self-tanning agents which can be employed are, inter alia:

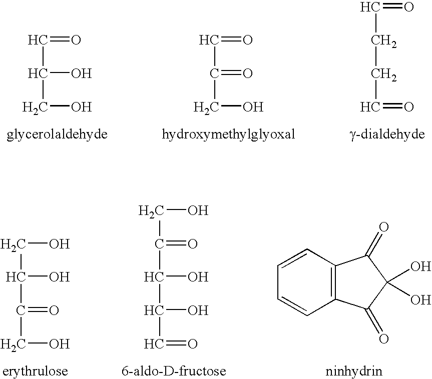

Mention should also be made of 5-hydroxy-1,4-naphthoquinone (juglone), which is extracted from the shells of fresh walnuts 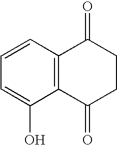
5-hydroxy-1,4-naphthoquinone (juglone)
and 2-hydroxy-1,4-naphthoquinone (lawsone), which occurs in henna leaves. 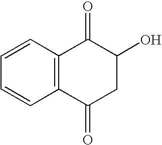
2-hydroxy-1,4-naphthoquinone (lawsone)

Very particular preference is given to 1,3-dihydroxyacetone (DHA), a tri-functional sugar which occurs in the human body, and derivatives thereof. 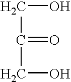
1,3-dihydroxyacetone (DHA)

Furthermore, the compositions according to the invention may also comprise dyes and coloured pigments. The dyes and coloured pigments can be selected from the corresponding positive list in the German Cosmetics Regulation or the EC list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | green |
| Acid Green 1 | 10020 | Green |
| 2,4-Dinitrohydroxynaphthalene-7-sulfonic acid | 10316 | Yellow |
| Pigment Yellow 1 | 11680 | Yellow |
| Pigment Yellow 3 | 11710 | Yellow |
| Pigment Orange 1 | 11725 | Orange |
| 2,4-Dihydroxyazobenzene | 11920 | Orange |
| Solvent Red 3 | 12010 | Red |
| 1-(2′-Chloro-4′-nitro-1′-phenylazo)-2-hydroxynaphthalene | 12085 | Red |
| Pigment Red 3 | 12120 | Red |
| Ceres Red; Sudan Red; Fat Red G | 12150 | Red |
| Pigment Red 112 | 12370 | Red |
| Pigment Red 7 | 12420 | Red |
| Pigment Brown 1 | 12480 | Brown |
| 4-(2′-Methoxy-5′sulfonyldiethylamide-1′-phenylazo)-3- | 12490 | Red |
| hydroxy-5″-chloro-2″,4″-dimethoxy2-naphthanilide | | |
| Disperse Yellow 16 | 12700 | Yellow |
| 1-(4-Sulfo-1-phenylazo)-4-aminobenzene-5-sulfonic acid | 13015 | Yellow |
| 2,4-Dihydroxyazobenzene-4′-sulfonic acid | 14270 | Orange |
| 2-(2,4-Dimethylphenylazo-5-sulfonyl)-1-hydroxynaphthalene- | 14700 | Red |
| 4-sulfonic acid | | |
| 2-(4-Sulfo-1-naphthylazo)-1-naphthol-4-sulfonic acid | 14720 | Red |
| 2-(6-Sulfo-2,4-xylylazo)-1-naphthol-5-sulfonic acid | 14815 | Red |
| 1-(4′-Sulfophenylazo)-2-hydroxynaphthalene | 15510 | Orange |
| 1-(2-Sulfonyl-4-chloro-5-carboxy-1-phenylazo)-2- | 15525 | Red |
| hydroxynaphthalene | | |
| 1-(3-Methylphenylazo-4-sulfonyl)-2-hydroxynaphthalene | 15580 | Red |
| 1-(4′,(8′)-Sulfonylnaphthylazo)-2-hydroxynaphthalene | 15620 | Red |
| 2-Hydroxy-1,2′-azonaphthalene-1′-sulfonic acid | 15630 | Red |
| 3-Hydroxy-4-phenylazo-2-naphthylcarboxylic acid | 15800 | Red |
| 1-(2-Sulfo-4-methyl-1-phenylazo)-2-naphthylcarboxylic | 15850 | Red |
| acid | | |
| 1-(2-Sulfo-4-methyl-5-chloro-1-phenylazo)-2-hydroxynaphthalene- | 15865 | Red |
| 3-carboxylic acid | | |
| 1-(2-Sulfo-1-naphthylazo)-2-hydroxynaphthalene-3- | 15880 | red |
| carboxylic acid | | |
| 1-(3-Sulfo-1-phenylazo)-2-naphthol-6-sulfonic acid | 15980 | Orange |
| 1-(4-Sulfo-1-phenylazo)-2-naphthol-6-sulfonic acid | 15985 | Yellow |
| Allura Red | 16035 | Red |
| 1-(4-Sulfo-1-naphthylazo)-2-naphthol-3,6-disulfonic acid | 16185 | Red |
| Acid Orange 10 | 16230 | Orange |
| 1-(4-Sulfo-1-naphthylazo)-2-naphthol-6,8-disulfonic acid | 16255 | Red |
| 1-(4-Sulfo-1-naphthylazo)-2-naphthol-3,6,8-trisulfonic | 16290 | Red |
| acid | | |
| 8-Amino-2-phenylazo-1-naphthol-3,6-disulfonic acid | 17200 | Red |
| Acid Red 1 | 18050 | Red |
| Acid Red 155 | 18130 | Red |
| Acid Yellow 121 | 18690 | Yellow |
| Acid Red 180 | 18736 | Red |
| Acid Yellow 11 | 18820 | Yellow |
| Acid Yellow 17 | 18965 | Yellow |
| 4-(4-Sulfo-1-phenylazo)-1-(4-sulfophenyl)-5-hydroxypyrazolone- | 19140 | Yellow |
| 3-carboxylic acid | | |
| Pigment Yellow 16 | 20040 | Yellow |
| 2,6-(4′-Sulfo-2″,4″-dimethyl)bisphenylazo)1,3-dihydroxybenzene | 20170 | Orange |
| Acid Black 1 | 20470 | Black |
| Pigment Yellow 13 | 21100 | Yellow |
| Pigment Yellow 83 | 21108 | Yellow |
| Solvent Yellow | 21230 | Yellow |
| Acid Red 163 | 24790 | Red |
| Acid Red 73 | 27290 | Red |
| 2-[4′-(4″-Sulfo-1″-phenylazo)-7′-sulfo-1′-naphthylazo]-1- | 27755 | black |
| hydroxy-7-aminonaphthalene-3,6-disulfonic acid | | |
| 4-[4″-Sulfo-1″-phenylazo)-7′-sulfo-1′-naphthylazo]-1- | 28440 | Black |
| hydroxy-8-acetylaminonaphthalene-3,5-disulfonic acid | | |
| Direct Orange 34, 39, 44, 46, 60 | 40215 | Orange |
| Food Yellow | 40800 | Orange |
| trans-β-Apo-8′-carotene aldehyde (C₃₀) | 40820 | Orange |
| trans-Apo-8′-carotinic acid (C₃₀) ethyl ester | 40850 | Orange |
| Canthaxanthine | 40850 | Orange |
| Acid Blue 1 | 42045 | Blue |
| 2,4-Disulfo-5-hydroxy-4′-4″- | 42051 | Blue |
| bis(diethylamino)triphenylcarbinol | | |
| 4-[(-4-N-Ethyl-p-sulfobenzylamino)phenyl-(4-hydroxy-2- | 42053 | Green |
| sulfophenyl)(methylene)-1-(N-ethylN-p-sulfobenzyl)-2,5- | | |
| cyclohexadienimine] | | |
| Acid Blue 7 | 42080 | Blue |
| (N-Ethyl-p-sulfobenzylamino)phenyl-(2-sulfophenyl)- | 42090 | Blue |
| methylene-(N-ethyl-N-p-sulfobenzyl)Δ^{2,5}- | | |
| cyclohexadienimine | | |
| Acid Green 9 | 42100 | Green |
| Diethyldisulfobenzyldi-4-amino-2-chlorodi-2- | 42170 | Green |
| methylfuchsonimmonium | | |
| Basic Violet 14 | 42510 | Violet |
| Basic Violet 2 | 42520 | Violet |
| 2′-Methyl-4′-(N-ethyl-N-m-sulfobenzyl)amino-4″-(N- | 42735 | Blue |
| diethyl)-amino-2-methyl-N-ethylN-m- | | |
| sulfobenzylfuchsonimmonium | | |
| 4′-(N-Dimethyl)amino-4″-(N-phenyl)aminonaphtho-N- | 44045 | Blue |
| dimethylfuchsonimmonium | | |
| 2-Hydroxy-3,6-disulfo-4,4′-bisdimethylaminonaphthofuchsonimmonium | 44090 | Green |
| Acid Red 52 | 45100 | Red |
| 3-(2′-Methylphenylamino)-6-(2′-methyl-4′- | 45190 | Violet |
| sulfophenylamino)-9-(2″-carboxyphenyl)xanthenium salt | | |
| Acid Red 50 | 45220 | Red |
| Phenyl-2-oxyfluorone-2-carboxylic acid | 45350 | yellow |
| 4,5-Dibromofluorescein | 45370 | Orange |
| 2,4,5,7-Tetrabromofluorescein | 45380 | Red |
| Solvent Dye | 45396 | Orange |
| Acid Red 98 | 45405 | Red |
| 3′,4′,5′,6′-Tetrachloro-2,4,5,7-tetrabromofluorescein | 45410 | Red |
| 4,5-Diiodofluorescein | 45425 | Red |
| 2,4,5,7-Tetraiodofluorescein | 45430 | Red |
| Quinophthalone | 47000 | Yellow |
| Quinophthalonedisulfonic acid | 47005 | Yellow |
| Acid Violet 50 | 50325 | Violet |
| Acid Black 2 | 50420 | Black |
| Pigment Violet 23 | 51319 | Violet |
| 1,2-Dioxyanthraquinone, calcium/aluminium complex | 58000 | Red |
| 3-Oxypyrene-5,8,10-sulfonic acid | 59040 | Green |
| 1-Hydroxy-4-N-phenylaminoanthraquinone | 60724 | Violet |
| 1-Hydroxy-4-(4′-methylphenylamino)anthraquinone | 60725 | Violet |
| Acid Violet 23 | 60730 | Violet |
| 1,4-Di(4′-methylphenylamino)anthraquinone | 61565 | Green |
| 1,4-Bis(o-sulfo-p-toluidino)anthraquinone | 61570 | Green |
| Acid Blue 80 | 61585 | Blue |
| Acid Blue 62 | 62045 | Blue |
| N,N′-Dihydro-1,2,1′,2′-anthraquinonazine | 69800 | Blue |
| Vat Blue 6; Pigment Blue 64 | 69825 | Blue |
| Vat Orange 7 | 71105 | orange |
| Indigo | 73000 | Blue |
| Indigodisulfonic acid | 73015 | Blue |
| 4,4′-Dimethyl-6,6′-dichlorothioindigo | 73360 | Red |
| 5,5′Dichloro-7,7′-dimethylthioindigo | 73385 | violet |
| Quinacridone Violet 19 | 73900 | violet |
| Pigment Red 122 | 73915 | Red |
| Pigment Blue 16 | 74100 | blue |
| Phthalocyanines | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopene | 75125 | yellow |
| trans-alpha-, -beta- or -gamma-Carotene | 75130 | orange |
| Keto and/or hydroxyl derivatives of carotene | 75135 | yellow |
| Guanine or pearlescent agent | 75170 | white |
| 1,7-Bis(4-hydroxy-3-methoxyphenyl)1,6-heptadiene-3,5- | 75300 | yellow |
| dione | | |
| Complex salt (Na, Al, Ca) of carminic acid | 75470 | Red |
| Chlorophyll a and b; copper compounds of chlorophylls | 75810 | green |
| and chlorophyllines | | |
| Aluminium | 77000 | white |
| Aluminium hydroxide | 77002 | white |
| Water-containing aluminium silicates | 77004 | white |
| Ultramarine | 77007 | blue |
| Pigment Red 101 and 102 | 77015 | Red |
| Barium sulfate | 77120 | white |
| Bismuth oxychloride and mixtures thereof with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, water-containing | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7 H₂O | 77745 | red |
| Silver | 77820 | white |
| Titanium dioxide and mixtures thereof with mica | 77891 | white |
| Zinc oxide | 77947 | white |
| 6,7-Dimethyl-9-(1′-D-ribityl)isoalloxazine, lactoflavin | | yellow |
| Sugar dye | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol Blue | | blue |

It may furthermore be favourable to select, as dye, one or more substances from the following group:
2,4-dihydroxyazobenzene, 1-(2′-chloro-4′-nitro-1′phenylazo)-2-hydroxy-naphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising pearlescent pigments. Particular preference is given to the types of pearlescent pigment listed below:
1. Natural pearlescent pigments, such as, for example,
   1. “pearl essence” (guanine/hypoxanthine mixed crystals from fish scales) and
   2. “mother-of-pearl” (ground mussel shells)
2. Monocrystalline pearlescent pigments, such as, for example, bismuth oxychloride (BiOCl)
3. Layered substrate pigments: for example mica/metal oxide

The basis for pearlescent pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| Group | thickness | Colour |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck under the trade names Timiron, Colorona or Dichrona.

The list of the said pearlescent pigments is of course not intended to be limiting. Pearlescent pigments which are advantageous for the purposes of the present invention can be obtained by numerous routes known per se. For example, other substrates apart from mica can also be coated with further metal oxides, such as, for example, silica and the like. For example, TiO₂- and Fe₂O₃-coated SiO₂ particles (“Ronasphere” grades), which are marketed by Merck and are particularly suitable for the optical reduction of fine wrinkles, are advantageous.

It may additionally be advantageous to completely omit a substrate such as mica. Particular preference is given to pearlescent pigments prepared using SiO₂. Such pigments, which may additionally also have goniochromatic effects, are available, for example, from BASF under the trade name Sicopearl Fantastico.

It may also be advantageous to employ Engelhard/Mearl pigments based on calcium sodium borosilicate coated with titanium dioxide. These are available under the name Reflecks. Due to their particle size of 40-80 μm, they have a glitter effect in addition to the colour.

Also particularly advantageous are effect pigments available from Flora Tech under the trade name Metasomes Standard/Glitter in various colours (yellow, red, green, blue). The glitter particles here are in the form of mixtures with various assistants and dyes (such as, for example, the dyes with the Colour Index (CI) numbers 19140, 77007, 77289, 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably 0.5 to 15% by weight, in particular 1.0 to 10% by weight, in each case based on the total weight of the compositions.

All compounds or components which can be used in the compositions are either known or commercially available or can be synthesised by known processes.

The one or more compounds of the formula I can be incorporated into cosmetic or dermatological compositions in the customary manner. Suitable compositions are those for external use, for example in the form of a cream, lotion or gel or as a solution which can be sprayed onto the skin. Suitable for internal use are administration forms such as capsules, coated tablets, powders, tablet solutions or solutions.

Use forms of the compositions according to the invention that may be mentioned are, for example, solutions, suspensions, emulsions, PIT emulsions, pastes, ointments, gels, creams, lotions, powders, soaps, surfactant-containing cleansing preparations, oils, aerosols and sprays. Examples of other use forms are sticks, shampoos and shower compositions. Any desired customary vehicles, assistants and, if desired, further active ingredients may be added to the composition.

Preferred assistants originate from the group of the preservatives, anti-oxidants, stabilisers, solubilisers, vitamins, colorants and odour improvers.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic use forms are also lipsticks, lip-care sticks, mascara, eyeliner, eye-shadow, rouge, powder make-up, emulsion make-up and wax make-up, and sunscreen, pre-sun and after-sun preparations.

The preferred composition forms according to the invention include, in particular, emulsions.

Emulsions according to the invention are advantageous and comprise, for example, the said fats, oils, waxes and other fatty substances, as well as water and an emulsifier, as usually used for a composition of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of the esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of the esters of aromatic carboxylic acids and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group of the branched and unbranched hydrocarbons and waxes, silicone oils, dialkyl ethers, or the group of the saturated and unsaturated, branched and unbranched alcohols, and fatty acid triglycerides, specifically the triglycerol esters of saturated and/or unsaturated, branched and/or unbranched alkanecarboxylic acids having a chain length of 8 to 24, in particular 12-18, C atoms. The fatty acid triglycerides may advantageously be selected, for example, from the group of the synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane or poly(methylphenylsiloxane).

Also particularly advantageous are mixtures of cyclomethicone and isotridecyl isononanoate and of cyclomethicone and 2-ethylhexyl isostearate.

The aqueous phase of the compositions according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the the polyacrylates, preferably a polyacrylate from the group of the the so-called Carbopols, for example Carbopol grades 980, 981, 1382, 2984 or 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the compositions according to the invention comprise hydrophilic surfactants.

The hydrophilic surfactants are preferably selected from the group of the the alkylglucosides, acyl lactylates, betaines and coconut amphoacetates.

The alkylglucosides are themselves advantageously selected from the group of the the alkylglucosides which are distinguished by the structural formula 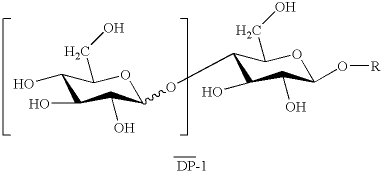
where R is a branched or unbranched alkyl radical having 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in per cent by weight. Products which are advantageous according to the invention are those having degrees of glucosylation of 1-2, particularly advantageously of 1.1 to 1.5, very particularly advantageously of 1,2-1.4, in particular of 1.3.

The value DP takes into account the fact that alkylglucosides are generally, as a consequence of their preparation, in the form of mixtures of mono- and oligoglucosides. A relatively high content of monoglucosides, typically in the order of 40-70% by weight, is advantageous in accordance with the invention.

Alkylglycosides which are particularly advantageously used for the purposes of the invention are selected from the group octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and assistants or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group of the the substances which are distinguished by the structural formula 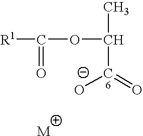
where R¹ is a branched or unbranched alkyl radical having 1 to 30 carbon atoms, and M⁺ is selected from the group of the the alkali metal ions and the group of the ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group of the the substances which are distinguished by the structural formula 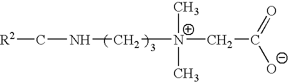
where R² is a branched or unbranched alkyl radical having 1 to 30 carbon atoms.

R² is particularly advantageously a branched or unbranched alkyl radical having 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The compositions according to the invention are advantageously characterised in that the hydrophilic surfactant(s) is (are) present in concentrations of 0.01-20% by weight, preferably 0.05-10% by weight, particularly preferably 0,1-5% by weight, in each case based on the total weight of the composition.

For use, the cosmetic and dermatological compositions are applied in sufficient amount to the skin and/or hair in the usual manner for cosmetics.

Cosmetic and dermatological compositions according to the invention may exist in various forms. Thus, they may be, for example, a solution, a water-free composition, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoines in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention.

Co-emulsifiers which are advantageous according to the invention are, for example, O/W emulsifiers, principally from the group of the the substances having HLB values of 11-16, very particularly advantageously having HLB values of 14.5-15.5, so long as the O/W emulsifiers have saturated radicals R and R′. If the ONw emulsifiers have unsaturated radicals R and/or R′ or in the case of isoalkyl derivatives, the preferred HLB value of such emulsifiers may also be lower or higher.

It is advantageous to select the fatty alcohol ethoxylates from the group of the ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:
polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-1 1 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention can be the following:
fatty alcohols having 8 to 30 C atoms, monoglycerol esters of saturated and/or unsaturated, branched and/or unbranched alkanecarboxylic acids having a chain length of 8 to 24 C atoms, in particular 12-18 C atoms, diglycerol esters of saturated and/or unsaturated, branched and/or unbranched alkanecarboxylic acids having a chain length of 8 to 24 C atoms, in particular 12-18 C atoms, monoglycerol ethers of saturated and/or unsaturated, branched and/or unbranched alcohols having a chain length of 8 to 24 C atoms, in particular 12-18 C atoms, diglycerol ethers of saturated and/or unsaturated, branched and/or unbranched alcohols having a chain length of 8 to 24 C atoms, in particular 12-18 C atoms, propylene glycol esters of saturated and/or unsaturated, branched and/or unbranched alkanecarboxylic acids having a chain length of 8 to 24 C atoms, in particular 12-18 C atoms, and sorbitan esters of saturated and/or unsaturated, branched and/or unbranched alkanecarboxylic acids having a chain length of 8 to 24 C atoms, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

Preferred compositions according to the invention are particularly suitable for protecting human skin against ageing processes and against oxidative stress, i.e. against damage by free radicals, as are produced, for example, by sunlight, heat or other influences. In this connection, they are in the various administration forms usually used for this application. For example, they may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The composition may comprise cosmetic adjuvants which are usually used in this type of composition, such as, for example, thickeners, softeners, moisturisers, surfactants, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which, apart from the compound(s) of the formula I or formula II, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The composition according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photochemical damage in order to prevent changes of colour shade, bleaching or damage of a mechanical nature. In this case, a suitable formulation is in the form of a rinse-out shampoo, lotion, gel or emulsion, the composition in question being applied before or after shampooing, before or after colouring or bleaching or before or after permanent waving. It is also possible to select a composition in the form of a lotion or gel for styling or treating the hair, in the form of a lotion or gel for brushing or blowwaving, in the form of a hair lacquer, permanent waving composition, colorant or bleach for the hair. Besides the compound(s) of the formula I or formula II, the composition having light-protection properties may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The present invention furthermore relates to a process for the preparation of a composition which is characterised in that at least one compound of the formula I or formula II containing radicals as described above is mixed with a cosmetically or dermatologically or food-suitable vehicle, and to the use of a compound of the formula I or formula II for the preparation of a composition.

The compositions according to the invention can be prepared here with the aid of techniques which are well known to the person skilled in the art.

The mixing can result in dissolution, emulsification or dispersal of the compound of the formula I or formula II in the vehicle.

It has also been noted that compounds of the formula I or formula II can have a stabilising effect on the composition. When used in corresponding products, the latter are thus also stable for longer and do not change their appearance. In particular, the effectiveness of the ingredients, for example vitamins, is retained even in the case of application over extended periods or extended storage. This is, inter alia, particularly advantageous in the case of compositions for protecting the skin against the effect of UV rays since these cosmetics are exposed to particularly high stresses by UV radiation.

The positive effects of compounds of the formula I or formula II give rise to their particular suitability for use in cosmetic or pharmaceutical compositions.

The properties of compounds of the formula I or formula II should likewise be regarded as positive for use in foods or as food supplements or as functional foods. The further explanations given for foods also apply correspondingly to food supplements and functional foods.

The foods which can be enriched with one or more compounds of the formula I or formula II in accordance with the present invention include all materials which are suitable for consumption by animals or consumption by humans, for example vitamins and provitamins thereof, fats, minerals or amino acids. (The foods may be solid, but also liquid, i.e. in the form of a beverage).

The present invention accordingly furthermore relates to the use of a compound of the formula I or formula II as food additive for human or animal nutrition, and to compositions which are foods or food supplements and comprise corresponding vehicles.

Foods which can be enriched with one or more compounds of the formula I or formula II in accordance with the present invention are, for example, also foods which originate from a single natural source, such as, for example, sugar, unsweetened juice, squash or puree of a single plant species, such as, for example, unsweetened apple juice (for example also a mixture of different types of apple juice), grapefruit juice, orange juice, apple compote, apricot squash, tomato juice, tomato sauce, tomato puree, etc. Further examples of foods which can be enriched with one or more compounds of the formula I or formula II in accordance with the present invention are corn or cereals from a single plant species and materials produced from plant species of this type, such as, for example, cereal syrup, rye flour, wheat flour or oat bran. Mixtures of foods of this type are also suitable for being enriched with one or more compounds of the formula I or formula II in accordance with the present invention, for example multivitamin preparations, mineral mixtures or sweetened juice. As further examples of foods which can be enriched with one or more compounds of the formula I or formula II in accordance with the present invention, mention may be made of food compositions, for example prepared cereals, biscuits, mixed drinks, foods prepared especially for children, such as yoghurt, diet foods, low-calorie foods or animal feeds.

The foods which can be enriched with one or more compounds of the formula I or formula II in accordance with the present invention thus include all edible combinations of carbohydrates, lipids, proteins, inorganic elements, trace elements, vitamins, water or active metabolites of plants and animals.

The foods which can be enriched with one or more compounds of the formula I or formula II in accordance with the present invention are preferably administered orally, for example in the form of meals, pills, tablets, capsules, powders, syrup, solutions or suspensions.

The foods according to the invention enriched with one or more compounds of the formula I or formula II can be prepared with the aid of techniques which are well known to the person skilled in the art.

Furthermore, compounds of the formula I have only a weak inherent colour. The weak inherent colour is, for example, a major advantage if an inherent colour of the ingredients is undesired in the products for aesthetic reasons.

The proportion of the compounds of the formula I in the composition is preferably 0.01 to 20% by weight, particularly preferably 0.05 to 10% by weight and especially preferably 0.1 to 5% by weight, based on the composition as a whole. The proportion of the compounds of the formula I in the composition is very particularly preferably 0.1 to 2% by weight, based on the composition as a whole.

Even without further comments, it is assumed that a person skilled in the art will be able to utilise the above description in the broadest scope. The preferred embodiments should therefore merely be regarded as descriptive disclosure which is absolutely not limiting in any way. The complete disclosure content of all applications and publications mentioned above and below is incorporated into this application by way of reference. The following examples are intended to illustrate the present invention. However, they should in no way be regarded as limiting. All compounds or components which can be used in the compositions are either known and commercially available or can be synthesised by known methods. The INCI names of the raw materials used are as follows:

### EXAMPLES

### Examples

### Example 1

### Preparation of 2-ethoxycarbonyl-7-hydroxychromone

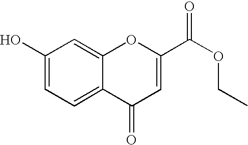

Sodium (7.6 g, 330 mmol) is initially introduced under an Ar atmosphere, and ethanol (500 ml) is slowly added dropwise. The mixture is stirred for approximately a further 1 hour until the sodium has completely dissolved and is subsequently cooled to RT using an ice bath. 2′,4′-Dihydroxyacetophenone (10 g, 66 mmol) and diethyl oxalate (36 ml, 266 mmol) dissolved in 60 ml of EtOH (brown-orange clear solution) are added dropwise. The solution is stirred at 70° C. for 2 hours. The clear solution is cooled to 0° C. using an ice/water bath and adjusted from pH 13 to pH 4 using about 50 ml of HCl (c=32%). Some of the ethanol is then removed from the suspension under reduced pressure. The remaining suspension is added to 300 ml of ice-water and extracted with CH₂Cl₂, the aqueous phase is extracted by shaking 2× with CH₂Cl₂, the org. phases are combined, extracted 3× with deionised water and 1× with saturated NaCl solution, and the org. phase is dried using Na sulfate, filtered and evaporated to dryness. Yield: 29.1 g of red-brown slurry-like solid.

100 ml of acetic acid and 1 ml of conc. sulfuric acid are added to the crude product, and the mixture is refluxed for 2 hours with stirring and cooled, and the solid which precipitates in the process is filtered off via a suction filter, washed with a little CH₃COOH and subsequently with deionised water until neutral and dried overnight in a vacuum drying cabinet at 40° C. and 200 mbar.

Yield: 10.1 g =65.6% of theory of pale-pink pulverulent solid.

Recrystallisation is carried out from a mixture of toluene and methanol.

Yield: 6.6 g=42.9% of theory of beige, fine crystals (HPLC=100%).

¹H NMR (300 MHz) in DMSO δ (ppm): 1.35 (t, 3H), 4.37 (q, 2H), 6.84 (s, 1H), 6.9 (d, 1H), 6.96 (dd, 1H), 7.9 (d, 1H), 11.0 (bs, 1OH).

MS (m/e): 234 (M⁺)

### Example 2

### Preparation of 7-hydroxy-4-oxo-4H-chromone-2-carboxylic acid

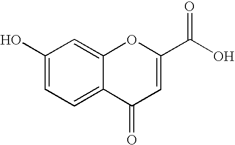

2-Ethoxycarbonyl-7-hydroxychromone (14.5 g, 62 mmol) is initially introduced dissolved in ethanol (400 ml) at 50° C, and sodium carbonate (20 g, 190 mmol) dissolved in deionised H₂O (200 ml) is added dropwise. The mixture is refluxed at 80° C. for 3 hours with stirring. After cooling, the mixture is acidified using 2N HCl. The precipitated white solid is filtered off with suction, washed until neutral and dried.

Yield: 6.5 g=50.9% of theory of a virtually white powder

¹H NMR (300 MHz) in DMSO δ (ppm): 6.8 (s, 1H), 6.9 (d, 1H), 6.95 (dd, 1H), 7.9 (d, 1H), 11.0 (bs, 1OH), 14.5 (bs, 1COOH)

MS (mle): 206 (M⁺)

### Example 2a

### Preparation of 1-ethylhexyl 7-hydroxy-4-oxo-4H-chromone-2-carboxylate

The ester is obtained by transesterification of the acid from Example 2 using 1-ethylhexyl alcohol.

¹H NMR (300 MHz) in CDCl₃ δ (ppm): 0.79-0.88 (m, 6H), 1.18-1.37 (m, 8H), 1.65 (ddd, 1H), 7.02-7.06 (m, 1H+2H arom.), 8.02 (d, 1H arom.)

### Example 3

### Preparation of 2-methoxyl-7-hydroxy-4H-chromen-4-one

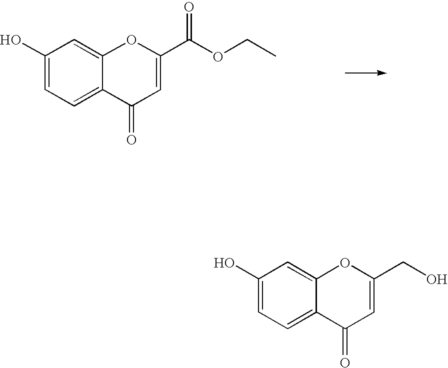

7-Hydroxychromen-4-on-2-ethoxycarbonyl (2 g-8,538 mmol) and granulated and dried calcium chloride (1 g-9.01 mmol) is initially introduced, and ethanol (absolute-40 ml) is added. Sodium borohydride (1.33 g-35.157 mmol) is subsequently added in portions with ice-cooling. The reaction mixture is stirred at RT for 2 h, then again cooled using an ice bath, and sodium borohydride (0.45 g-11.895 mmol) is again added. The mixture is stirred overnight at RT.

The ethanol is subsequently removed in a rotary evaporator (bath temperature: 50° C.), 60 ml of deionised water are carefully added to the residue, and the suspension is acidified dropwise using 2N HCl. About 100 g of ice are subsequently added to the solution, and the mixture is stirred for half an hour, during which a white solid precipitated, which is filtered off with suction and dried in a vacuum drying cabinet at 45° C. 1.1 g of white solid. Yield: 67%

¹H NMR in DMSO δ (ppm): 4.4 (s, 2H), 6.2 (s, 1H), 6.8 (d, 1H), 6.9 (dd, 1H), 7.9 (d, 1H).

MS (m/e): 192 (M⁺);

### Example 4

### Preparation of 5,7-dihydroxy-4-oxo-4H-chromene-2-carboxylic acid

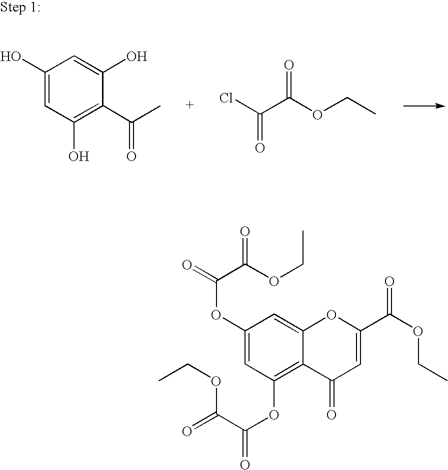

2,4,6-Trihydroxyacetophenone dissolved in pyridine is initially introduced under an argon atmosphere, and a little 4-(dimethylamino)pyridine (catalytic amount) is introduced. The ethyl chloroformylformate is then slowly added dropwise. When everything has been added, the apparatus is heated to 80° C. using an oil bath and stirred at this temperature for 2 hours.

The apparatus is allowed to cool to room temperature, the dark-brown suspension is added to about 200 ml of ice-water, 200 ml of CH₂Cl₂ are added, and the mixture is extracted. The aqueous phase is extracted by shaking a further 2× with 50 ml of CH₂Cl₂, and the black org. phases are combined and washed 2× with 50 ml of deionised water, 3× with 2 molar HCl (pyridine-free) and 1× with saturated NaCl solution, leaving a clear black-brown org. phase, which is dried using Na₂SO₄. The organic phase is passed through a glass frit with a little silica gel # 7734 slurried in CH₂Cl₂/EEE (5:1), the filter cake is rinsed with about 250 ml of CH₂Cl₂/EEE (5:1), and the solution is evaporated in a rotary evaporator. Yield: 8.5 g of yellow solid. This solid is used as it is for the next step.
Step 2:

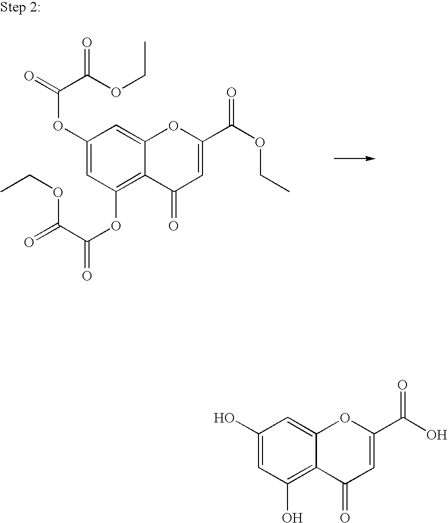

2-Ethoxycarbonyl-7-ethoxyoxalyloxy-4-oxo-4H-chromen-5-yl ethyl oxalate from Step 1 dissolved in ethanol is initially introduced at room temperature, and Na₂CO₃ dissolved in deionised H₂O is added dropwise. The mixture is subsequently heated to 70° C. and stirred at this temperature for a further 4 hours. After cooling, 100 ml of ethyl acetate are added to the reaction mixture, which is slightly acidified using 1N HCl. The aqueous phase is separated off and extracted. The org. phases are combined, washed 3× with deionised H₂O and 1× with sat. NaCl solution, dried using Na₂SO₄, filtered and evaporated in a rotary evaporator. Recrystallisation gives 0.4 g of yellow fine crystals (HPLC=98.4%).

¹H NMR (300 MHz) in DMSO δ (ppm): 6.2 (d, 1H), 6.4 (d, 1H), 6.8 (s, 1H), 11.1 (bs, 1H), 12.5 (bs, 1H)

MS (m/e): 222 (M⁺)

### Example 4a

### Preparation of 1-ethylhexyl 5,7-dihydroxy-4-oxo-4H-chromene-2-carboxylate

The ester is obtained by transesterification of the acid from Example 3 using 1-ethylhexyl alcohol.

### Example 5

### Preparation of 5,7-diacetoxy-3-acetyl-2-methylchromen-4-one

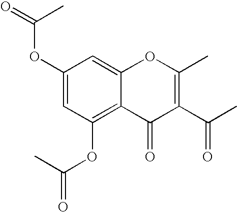

2,4,6-Trihydroxyacetophenone dissolved in acetic anhydride is initially introduced, and sodium acetate is added. The suspension is refluxed with stirring for 10 hours. The reaction mixture is subsequently poured into about 300 ml of ice-water and extracted 2× with ethyl acetate (EA), and the org. phases are combined and washed 3× with deionised H₂O. The solution which remains is washed further with Na₂HCO₃ solution. The organic phase is dried over Na₂SO₄, filtered and evaporated in a rotary evaporator.

¹H NMR (300 MHz) in DMSO δ (ppm): 7.1 (d, 1 H), 7.4 (d, 1H)

MS (m/e): 318 (M⁺)

### Example 6

### Preparation of 5,7-dihydroxy-2-methylchromen-4-one

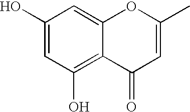

5,7-Diacetoxy-3-acetyl-2-methylchromen-4-one is boiled under reflux for 1 h with 40 ml of 10% sodium carbonate solution. After cooling, the suspension is adjusted to pH about 6 using 2N HCl and cooled. The precipitate is filtered off, giving 0.6 g of very pale-brown powder (T_{M}=279.9° C.)

¹H NMR (300 MHz) in DMSO δ (ppm): 2.3 (s, 3H), 6.15 (s, 1H), 6.18 (d, 1H), 6.3 (d, 1H), 10.8 (bs, 1OH), 12.8 (s, 1OH)

MS (m/e): 192 (M⁺)

### Example 7

### Preparation of 5,7-dihydroxy-2-ethylpentylchromen-4-one

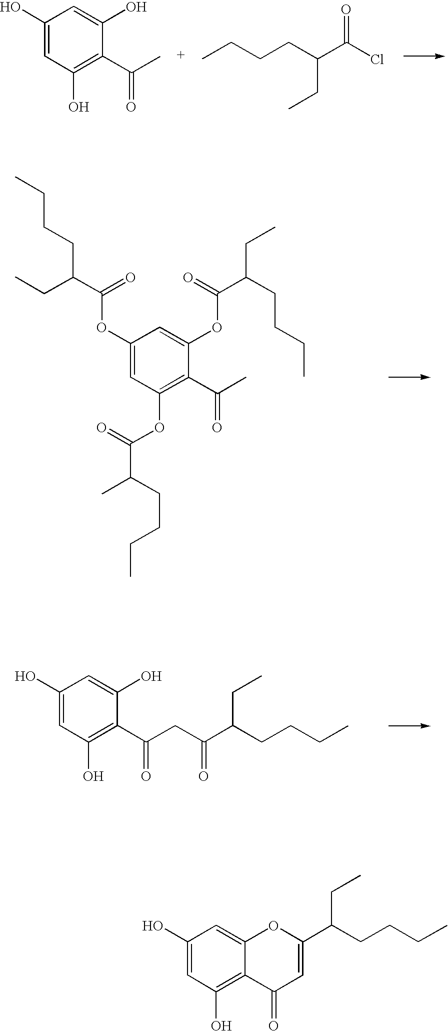 1st Step:

2,4,6-Trihydroxyacetophenone (5 g, 26.3 mmol) is added to 90 ml of toluene, and 14 g of potassium carbonate dissolved in 70 ml of deionised water and 1 g of tetra-n-butylammonium hydrogensulfate are added to the solution. 2-Ethylhexanoyl chloride (20.5 ml, 119.7 mmol) is added dropwise to the two-phase mixture over the course of 10 minutes with vigorous stirring. The two-phase mixture is subsequently heated at 70° C. for 5 hours with stirring.

The upper dark-red organic phase is subsequently separated off, the aqueous phase is extracted by shaking twice with dichloromethane, and the organic phases are combined, washed with saturated sodium chloride solution, dried over sodium sulfate, filtered and evaporated to dryness in a Rotavapor (bath temperature: 50° C.).

M(R): 19.3 g

2nd Step:

19.3 g of the product from the 1st step are dissolved in 600 ml of THF, and lithium hydroxide (4.4 g, 183.7 mmol) is added. The mixture is subsequently refluxed for 5.5 hours. The red-brown reaction solution is poured onto about 800 g of ice+100 ml of conc. HCl and extracted a number of times with dichloromethane, and the orange combined organic phases are washed with saturated sodium chloride solution, dried over sodium sulfate, filtered and evaporated to dryness in a rotary evaporator (bath temperature: 50° C.).

M(R): 17.2 g

3rd Step:

17.2 g of the product from the 2nd step are dissolved in 200 ml of acetic acid, and 2 ml of conc. sulfuric acid are added. The mixture is subsequently refluxed for 7 hours with stirring. The red-brown cloudy solution is poured onto about 500 g of ice, the red-brown precipitated solid is filtered off via a suction filter, taken up in dichloromethane and, together with the aqueous filtrate, extracted a number of times by shaking with dichloromethane, and the combined organic phases are washed with saturated sodium chloride solution, dried over sodium sulfate, filtered and evaporated to dryness in a rotary evaporator (bath temperature: 50° C.).

m(R): 18.4 g of residue, TLC: one spot

The residue is dissolved in a little methanol, and deionised water is added, whereupon a beige solid precipitates, which is filtered off via a small suction filter.

m(K): 1.65 g of beige solid

The filtrate is evaporated again, and 100 ml of heptane are added to the distillation residue, whereupon a solid precipitates, which is filtered off via a suction filter.

m(K2): 2.27 g of pale-brown solid

m(K tot.): 3.92 g are 52.3% of the theoretical yield, based on the amount of 2,4,6-trihydroxyacetophenone used.

¹H NMR (300 MHz) in DMSO δ (ppm): 0.9 (m, 6H), 1.15-1.3 (m, 4H), 1.55-1.65 (m, 4H), 2.45 (q, 1H), 6.17 (s, 1H), 6.2 (d, 1H), 6.35 (d, 1H), 10.75 (bs, OH), 12.85 (s, OH).

MS (m/e): 276 (M⁺)

The following is prepared analogously: 5,7-dihydroxy-3-(2-methoxyacetyl)-2-methoxymethylchromen-4-one

### Example 8

### Preparation of 7-isopropyl-4-oxo-4H-chromone-3-carbaldehyde

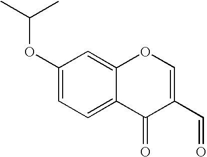

7-Hydroxy-4-oxo-4H-chromone-3-carbaldehyde (2 g, 10.5 mmol) is dissolved in N,N-dimethylformamide (25 ml) under an N₂ atmosphere, potassium carbonate (1.8 g, 13 mmol) and potassium iodide (50 mg) are added, and the mixture is stirred at RT for 1 hour. 2-Bromopropane (2 ml, 21 mmol) is then slowly added dropwise, and the mixture is heated at 55° C. for 2 hours. A further 2 ml of 2-bromopropane are added, and the mixture is stirred at 55° C. for a further 2.5 hours. After stirring at RT for 12 hours, the reaction mixture is introduced into 60 ml of deionised water, acidified using dilute HCl and extracted with 150 ml of EA. The aqueous phase is extracted a further 2× with EA. The combined org. phases are extracted by shaking 2× with 150 ml of deionised water and 1× with saturated NaCl solution, dried using Na sulfate and filtered, and the solvent is stripped off. For purification, the crude product is dissolved in 10 ml of eluent (CH₂Cl₂/MeOH 9.5/0.5) and filtered through 250 g of silica gel #109385. Yield: 281 mg=11.52% of theory. (HPLC content: 89.3%).

¹H NMR (300 MHz) in DMSO δ (ppm): 1.3 (d, 6H), 4.9 (m, 1H), 7.1 (dd, 1H), 7.3 (d, 1H), 8.85 (s, 1H), 10.1 (s, 1H).

### Example 9

### Preparation of L-ascorbyl 6-[5,7-dihydroxy-4-oxo-4H-chromone-2-carboxylate]

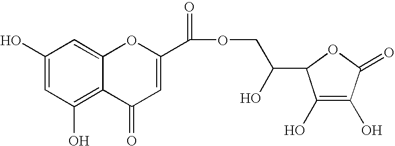

5,7-Dihydroxy-4-oxo-4H-chromone-2-carboxylic acid (400 mg, 1.8 mmol) dissolved in 95-97% sulfuric acid (10 ml) is initially introduced under an argon atmosphere and warmed to 55° C. Ten 100 mg portions of L-(+)-ascorbic acid are introduced slowly, during which the temperature is held at a maximum of 75° C. The mixture is subsequently stirred at this temperature for 12 hours.

The reaction mixture is cooled using an ice bath and introduced into 50 ml of ice-water, EA is added, the mixture is filtered through Celite, the aqueous phase is separated off and extracted again with a little EA, and the org. phases are combined, washed 4× with about 20 ml of deionised H₂O each time and 1× with sat. NaCl solution until neutral, dried using Na₂SO₄, filtered and evaporated in a rotary evaporator.

Yield: 250 mg

HPLC-ESI-MS shows [M+H]⁺=365.1

### Example 10

### Preparation of a Cyclodextrin Complex

3.4 g of hydroxypropyl-gamma-cyclodextrin (Aldrich; 2 ′-hydroxypropyl-cyclooctaamylose; Cas. No. 128446-34-4) are initially introduced in 25 ml of water and warmed to 50° C. 0.2 g of 5,7-dihydroxy-2-methylchromen-4-one (from Example 6) are dissolved in 25 ml of ethanol and added dropwise to the initially introduced solution. The solution is stirred overnight at 50° C. The ethanol is removed from the solution by distillation. The residue is evaporated to dryness under reduced pressure, and the solid which remains is dried further overnight at 40° C. and 200 mbar. Yield: 3.45 g

Characterisation:

Evidence of Complex Formation by Means of 2D NMR Spectrum

ROESY spectra show interaction of spatially adjacent atoms. Spatially close atoms give signals in the ROESY 2D NMR spectrum. Here, the complex was measured by means of ROESY in order to clarify the molecular constituents of 5,7-dihydroxy-2-methylchromen-4-one via which the complex formation takes place. In the ROESY spectrum (solvent D₂O), signals occur which can be assigned to an interaction of the atoms 6-H, 8-H and 2-CH₃ (cf. formula drawing) with the cyclodextrin molecules.

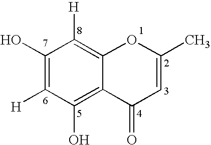

The NMR data fit the interpretation that a complex has formed which consists of 5,7-dihydroxy-2-methylchromen-4-one and two cyclodextrin molecules.
Content of 5,7-dihydroxy-2-methylchromen-4-one in the Solid (HPLC Determination)

5.4 mg of 5,7-dihydroxy-2-methylchromen-4-one are dissolved in 3 ml of methanol and 1 ml of THF and made up to 10.0 ml with eluent (acetonitrile/H₂O 2/8) in the volumetric flask (peak area of 21363731).

21.6 mg of complex are dissolved in 3 ml of methanol and 1 ml of tetrahydrofuran and made up to 10.0 ml with eluent (acetonitrile/H₂O 2/8) in the volumetric flask (peak area 5830414).

Conclusion: the complex consists of 6.8% by weight of 5,7-dihydroxy-2-methylchromen-4-one. A 5,7-dihydroxy-2-methylchromen-4-one: cyclodextrin weight ratio of about 6.8:93.2 is present in the complex. This corresponds to a molar ratio of about 1:2 (theoretical weight ratio of the 5,7-dihydroxy-2-methylchromen-4-one (cyclodextrin)₂ complex=5.7:94.3). The complex compound is a [5,7-dihydroxy-2-methylchromen-4-one]-[hydroxypropyl-gamma-cyclodextrin]₂ complex.

Solubility of the 5,7-dihydroxy-2-methylchromen-4-one/Cyclodextrin Complex:

0.5 g of complex is dissolved in 1 ml of water without reaching saturation. This corresponds to a solubility, based on pure 5,7-dihydroxy-2-methyl-chromen-4-one, of at least 34.5 mg/ml.

Cyclodextrin complexes of the chromone derivatives according to Examples 1-5 and 7-9 are prepared analogously to Example 10.

In the following example recipes, the name of the chromone derivative in each case stands for the corresponding hydroxypropyl-gamma-cyclodextrin complex, where the amount data are based on the chromone derivative.

### Example 11

Lotion (W/O) for application to the skin

| | | % by wt. |
|---|---|---|
| A | Polyglyceryl 2-dipolyhydroxystearate | 5.0 |
| | Beeswax | 0.5 |
| | Zinc stearate | 0.5 |
| | Hexyl laurate | 9.0 |
| | Cetyl isononanoate | 6.0 |
| | Shea butter | 0.5 |
| | DL-α-tocopherol acetate | 1.0 |
| | 5,7-Dihydroxy-2-methylchromen-4-one | 0.5 |
| B | Glycerol | 5.0 |
| | Magnesium sulfate heptahydrate | 1.0 |
| | Preservatives | q.s. |
| | Water, demineralised | to 100 |

Preparation

Phase A is warmed to 75° C. and phase B to 80° C. Phase B is slowly added to phase A with stirring. After homogenisation, the mixture is cooled with stirring. Perfumes are added at a temperature of 40° C.

The following preservatives are used:
0.05% of propyl 4-hydroxybenzoate
0.15% of methyl 4-hydroxybenzoate

### Example 12

Lotion (W/O) for application to the skin

| | | % by wt. |
|---|---|---|
| A | Polyglyceryl 2-dipolyhydroxystearate | 5.0 |
| | Beeswax | 0.5 |
| | Zinc stearate | 0.5 |
| | Hexyl laurate | 9.0 |
| | Cetyl isononanoate | 6.0 |
| | Shea butter | 0.5 |
| | DL-α-tocopherol acetate | 1.0 |
| B | 5,7-Dihydroxy-2-methylchromen-4-one | 1.0 |
| | Glycerol | 5.0 |
| | Magnesium sulfate heptahydrate | 1.0 |
| | Preservatives | q.s. |
| | Water, demineralised | to 100 |

Preparation

Phase A is warmed to 75° C. and phase B to 80° C. Phase B is slowly added to phase A with stirring. After homogenisation, the mixture is cooled with stirring. Perfumes are added at a temperature of 40° C.

The following preservatives are used:
0.05% of propyl 4-hydroxybenzoate
0.15% of methyl 4-hydroxybenzoate

### Example 13

Lotion (W/O) for application to the skin

| | | % by wt. |
|---|---|---|
| A | 4,6,3′,4′-Tetrahydroxybenzylcoumaranone-3 | 1.0 |
| | Polyglyceryl 2-dipolyhydroxystearate | 5.0 |
| | Beeswax | 0.5 |
| | Zinc stearate | 0.5 |
| | Hexyl laurate | 9.0 |
| | Cetyl isononanoate | 6.0 |
| | Shea butter | 0.5 |
| | DL-α-tocopherol acetate | 1.0 |
| | 5,7-Dihydroxy-2-methylchromen-4-one | 1.0 |
| B | Glycerol | 5.0 |
| | Magnesium sulfate heptahydrate | 1.0 |
| | Preservatives | q.s. |
| | Water, demineralised | to 100 |

Preparation

Phase A is warmed to 75° C. and phase B to 80° C. Phase B is slowly added to phase A with stirring. After homogenisation, the mixture is cooled with stirring. Perfumes are added at a temperature of 40° C.

The following preservatives are used:
0.05% of propyl 4-hydroxybenzoate
0.15% of methyl 4-hydroxybenzoate

### Example 14

A cream (O/W) comprising ectoine is prepared from the following components:

| | | | % by wt. |
|---|---|---|---|
| A | Paraffin, liquid | (1) | 8.0 |
| | Isopropyl myristate | (1) | 4.0 |
| | Mirasil CM5 | (2) | 3.0 |
| | Stearic acid | (1) | 3.0 |
| | Arlacel 165 V | (3) | 5.0 |
| | 5,7-Dihydroxy-2-methylchromen-4-one | | 1.0 |
| B | Glycerol (87%) | (1) | 3.0 |
| | Germaben II | (4) | 0.5 |
| | Water, demineralised | | to 100 |
| C | RonaCare ™ ectoine | (1) | 1.0 |

Preparation

Firstly, phases A and B are warmed separately to 75° C. Phase A is then slowly added to phase B with stirring, and the mixture is stirred until a homogeneous mixture has formed. After homogenisation of the emulsion, the mixture is cooled to 30° C. with stirring. The mixture is subsequently warmed to 35° C., phase C is added, and the mixture is stirred to homogeneity.

Sources of supply

| | |
|---|---|
| (1) | Merck KGaA |
| (2) | Rhodia |
| (3) | Uniqema |
| (4) | ISP |

### Example 15

Topical Composition as W/O Emulsion

| | | | % by wt. |
|---|---|---|---|
| A | Isolan PDI | (2) | 3.0 |
| | Paraffin oil, liq. | (1) | 17.0 |
| | Isopropyl myristate | | 5.0 |
| | Beeswax | | 0.2 |
| | Cutina HR | (2) | 0.3 |
| | 5,7-Dihydroxy-2-methylchromen-4-one | | 1.0 |
| B | Water, demineralised | | to 100 |
| | Glycerol (87%) | | 4.0 |
| | Magnesium sulfate | | 1.0 |
| | Germaben II-E | (3) | 1.0 |
| C | RonaCare ™ LPO | (1) | 2.0 |

Preparation

Phases A and B are warmed to 75° C. Phase B is added to phase A with stirring. The mixture is subsequently homogenised at 9000 rpm for 2 min. using the Turrax. The resultant mixture is cooled to 30 to 35° C., and C is stirred in.

Sources of supply

| | |
|---|---|
| (1) | Merck KGaA |
| (2) | Goldschmidt AG |
| (3) | ISP |

### Example 16

### Compositions

Illustrative recipes for cosmetic compositions which comprise chromen-4-one(2-hydroxypropyl-gamma-cyclodextrin) complexes according to Example 10 are indicated below. The name of the chromone derivative in each case stands for the corresponding hydroxypropyl-gamma-cyclodextrin complex, where the amount data are based on the chromone derivative. In addition, the INCI names of the commercially available compounds are indicated.

UV-Pearl, OMC stands for the composition having the INCI name: Water (for EU: Aqua), Ethylhexyl Methoxycinnamate, Silica, PVP, Chlorphenesin, BHT; this composition is commercially available under the name Eusolex® UV Pearl™ OMC from Merck KGaA, Darmstadt. The other UV-pearls indicated in the tables each have an analogous composition, with OMC being replaced by the UV filters indicated.

TABLE 1
W/O emulsions (data in % by weight)

| | 1-1 | 1-2 | 1-3 | 1-4 | 1-5 | 1-6 | 1-7 | 1-8 | 1-9 | 1-10 |
|---|---|---|---|---|---|---|---|---|---|---|
| Titanium Dioxide | | 2 | 5 | | | | | | | 3 |
| 2-Methyl-5,7-dihydroxy- | 5 | 3 | 2 | 1 | 2 | | | | 1 | 1 |
| chromen-4-one | | | | | | | | | | |
| 2-(1-Ethylhexyl)-5,7- | | | | | | 1 | 2 | 1 | | |
| dihydroxychromen-4-one | | | | | | | | | | |
| Zinc Oxide | | | | | | | | 5 | 2 | |
| UV-Pearl, OMC | 30 | 15 | 15 | 15 | 15 | 15 | 15 | 15 | 15 | 15 |
| Polyglyceryl-3-Dimerate | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Water | to | to | to | to | to | to | to | to | to | to |
| | 100 | 100 | 100 | 100 | 100 | 100 | 100 | 100 | 100 | 100 |

| | 1-11 | 1-12 | 1-13 | 1-14 | 1-15 | 1-16 | 1-17 | 1-18 |
|---|---|---|---|---|---|---|---|---|
| Titanium Dioxide | 3 | | 2 | | 3 | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 2-(1-Ethylhexyl)-5,7-dihydroxy- | 5 | 3 | 2 | 5 | 1 | 3 | 7 | 2 |
| chromen-4-one | | | | | | | | |
| Polyglyceryl-3-Dimerate | 3 | 3 | 3 | 3 | | | | |
| Cera Alba | 0.3 | 0.3 | 0.3 | 0.3 | 2 | 2 | 2 | 2 |
| Hydrogenated Castor Oil | 0.2 | 0.2 | 0.2 | 0.2 | | | | |
| Paraffinium Liquidum | 7 | 7 | 7 | 7 | | | | |
| Caprylic/Capric Triglyceride | 7 | 7 | 7 | 7 | | | | |
| Hexyl Laurate | 4 | 4 | 4 | 4 | | | | |
| PVP/Eicosene Copolymer | 2 | 2 | 2 | 2 | | | | |
| Propylene Glycol | 4 | 4 | 4 | 4 | | | | |
| Magnesium Sulfate | 0.6 | 0.6 | 0.6 | 0.6 | | | | |
| Tocopherol | 0.5 | 0.5 | 0.5 | 0.5 | | | | |
| Tocopheryl Acetate | 0.5 | 0.5 | 0.5 | 0.5 | 1 | 1 | 1 | 1 |
| Cyclomethicone | 0.5 | 0.5 | 0.5 | 0.5 | | | | |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Dicocoyl Pentyerythrityl Citrate (and) | | | | | 6 | 6 | 6 | 6 |
| Sorbitan Sesquioleate (and) Cera | | | | | | | | |
| Alba (and) Aluminium Stearate | | | | | | | | |
| PEG-7 Hydrogenated Castor Oil | | | | | 1 | 1 | 1 | 1 |
| Zinc Stearate | | | | | 2 | 2 | 2 | 2 |
| Oleyl Erucate | | | | | 6 | 6 | 6 | 6 |
| Decyl Oleate | | | | | 6 | 6 | 6 | 6 |
| Dimethicone | | | | | 5 | 5 | 5 | 5 |
| Tromethamine | | | | | 1 | 1 | 1 | 1 |
| Glycerine | | | | | 5 | 5 | 5 | 5 |
| Allantoin | | | | | 0.2 | 0.2 | 0.2 | 0.2 |
| Water | to | to | to | to | to | to | to | to |
| | 100 | 100 | 100 | 100 | 100 | 100 | 100 | 100 |

| | 1-19 | 1-20 | 1-21 | 1-22 | 1-23 | 1-24 | 1-25 | 1-26 | 1-27 | 1-28 | 1-29 |
|---|---|---|---|---|---|---|---|---|---|---|---|
| Titanium Dioxide | | 2 | 5 | | | | | | | 3 | 3 |
| Benzylidene Malonate Polysiloxane | | | | 1 | | | | | 1 | 1 | |
| Zinc Oxide | | | | | | | | 5 | 2 | | |
| 2-Methyl-5,7-dihydroxychromen-4- | 5 | 5 | 5 | 5 | 7 | 5 | 5 | 5 | 5 | 5 | 8 |
| one | | | | | | | | | | | |
| UV-Pearl, OCR | | 10 | | | | | | | | | 5 |
| UV-Pearl, EthylhexylDimethylPABA | | | 10 | | | | | | | | |
| UV-Pearl, Homosalate | | | | 10 | | | | | | | |
| UV-Pearl, Ethylhexyl Salicylate | | | | | 10 | | | | | | |
| UV-Pearl, OMC. BP-3 | | | | | | 10 | | | | | |
| UV-Pearl, OCR. BP-3 | | | | | | | 10 | | | | |
| UV-Pearl, Ethylhexyl Dimethyl | | | | | | | | 10 | | | |
| PABA, BP-3 | | | | | | | | | | | |
| UV-Pearl, Homosalate, BP-3 | | | | | | | | | 10 | | |
| UV-Pearl, Ethylhexyl Salicylate, | | | | | | | | | | 10 | |
| BP-3 | | | | | | | | | | | |
| BMDBM | | | | | | | | | | | 2 |
| UV-Pearl, OMC, | 25 | | | | | | | | | | |
| 4-Methylbenzylidene Camphor | | | | | | | | | | | |
| Polyglyceryl-3-Dimerate | 3 | 3 | 3 | 3 | 3 | 3 | 3 | 3 | 3 | 3 | 3 |
| Cera Alba | 0.3 | 0.3 | 0.3 | 0.3 | 0.3 | 0.3 | 0.3 | 0.3 | 0.3 | 0.3 | 0.3 |
| Hydrogenated Castor Oil | 0.2 | 0.2 | 0.2 | 0.2 | 0.2 | 0.2 | 0.2 | 0.2 | 0.2 | 0.2 | 0.2 |
| Paraffinium Liquidum | 7 | 7 | 7 | 7 | 7 | 7 | 7 | 7 | 7 | 7 | 7 |
| Caprylic/Capric Triglyceride | 7 | 7 | 7 | 7 | 7 | 7 | 7 | 7 | 7 | 7 | 7 |
| Hexyl Laurate | 4 | 4 | 4 | 4 | 4 | 4 | 4 | 4 | 4 | 4 | 4 |
| PVP/Eicosene Copolymer | 2 | 2 | 2 | 2 | 2 | 2 | 2 | 2 | 2 | 2 | 2 |
| Propylene Glycol | 4 | 4 | 4 | 4 | 4 | 4 | 4 | 4 | 4 | 4 | 4 |
| Magnesium Sulfate | 0.6 | 0.6 | 0.6 | 0.6 | 0.6 | 0.6 | 0.6 | 0.6 | 0.6 | 0.6 | 0.6 |
| Tocopherol | 0.5 | 0.5 | 0.5 | 0.5 | 0.5 | 0.5 | 0.5 | 0.5 | 0.5 | 0.5 | 0.5 |
| Tocopheryl Acetate | 0.5 | 0.5 | 0.5 | 0.5 | 0.5 | 0.5 | 0.5 | 0.5 | 0.5 | 0.5 | 0.5 |
| Cyclomethicone | 0.5 | 0.5 | 0.5 | 0.5 | 0.5 | 0.5 | 0.5 | 0.5 | 0.5 | 0.5 | 0.5 |
| Propylparaben | 0.05 | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 | 0.15 |

| | |
|---|---|
| Water | to 100 |

TABLE 2
O/W emulsions, data in % by weight

| | 2-1 | 2-2 | 2-3 | 2-4 | 2-5 | 2-6 | 2-7 | 2-8 | 2-9 | 2-10 |
|---|---|---|---|---|---|---|---|---|---|---|
| Titanium Dioxide | | 2 | 5 | | | | | | | 3 |
| Methylene Bis-benzotriazolyl | | | | | | 1 | 2 | 1 | | |
| Tetramethylbutylphenol | | | | | | | | | | |
| 2-(1-Ethylhexyl)-5,7-dihydroxy- | | | | 1 | 2 | | | | 1 | 1 |
| chromen-4-one | | | | | | | | | | |
| 4′-Methoxy-6-hydroxyflavone | 1 | 3 | | 2 | | 5 | | 5 | 2 | |
| 2-(Methoxy-methyl)-5,7- | 5 | 5 | 5 | 5 | 5 | 5 | 5 | 5 | 5 | 5 |
| dihydroxychromen-4-one | | | | | | | | | | |
| 2-Carboxy-5,7-dihydroxy- | 1 | 5 | 4 | | 6 | | 7 | | 2 | 1 |
| chromen-4-one | | | | | | | | | | |
| 4-Methylbenzylidene Camphor | 2 | | 3 | | 4 | | 3 | | 2 | |
| BMDBM | 1 | 3 | | 3 | 3 | | 3 | 3 | 3 | |
| Stearyl Alcohol (and) Steareth-7 | 3 | 3 | 3 | 3 | 3 | 3 | 3 | 3 | 3 | 3 |
| (and) Steareth-10 | | | | | | | | | | |
| Glyceryl Stearate (and) Ceteth- | 3 | 3 | 3 | 3 | 3 | 3 | 3 | 3 | 3 | 3 |
| 20 | | | | | | | | | | |
| Glyceryl Stearate | 3 | 3 | 3 | 3 | 3 | 3 | 3 | 3 | 3 | 3 |
| Microwax | 1 | 1 | 1 | 1 | 1 | 1 | 1 | 1 | 1 | 1 |
| Cetearyl Octanoate | 11.5 | 11.5 | 11.5 | 11.5 | 11.5 | 11.5 | 11.5 | 11.5 | 11.5 | 11.5 |
| Caprylic/Capric Triglyceride | 6 | 6 | 6 | 6 | 6 | 6 | 6 | 6 | 6 | 6 |
| Oleyl Oleate | 6 | 6 | 6 | 6 | 6 | 6 | 6 | 6 | 6 | 6 |
| Propylene Glycol | 4 | 4 | 4 | 4 | 4 | 4 | 4 | 4 | 4 | 4 |
| Glyceryl Stearate SE | | | | | | | | | | |
| Stearic Acid | | | | | | | | | | |
| Persea Gratissima | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Glycerine | | | | | | | | | | |
| Water | to | to | to | to | to | to | to | to | to | to |
| | 100 | 100 | 100 | 100 | 100 | 100 | 100 | 100 | 100 | 100 |

| | 2-11 | 2-12 | 2-13 | 2-14 | 2-15 | 2-16 | 2-17 | 2-18 |
|---|---|---|---|---|---|---|---|---|
| Titanium Dioxide | 3 | | 2 | | | | 2 | 5 |
| Benzylidene Malonate Polysiloxane | | 1 | 0.5 | | | | | |
| Methylene Bis-benzotriazolyl | 1 | 1 | 0.5 | | | | | |
| Tetramethylbutylphenol | | | | | | | | |
| 4′-Methoxy-7-β-glucoside Flavone | | | | 1 | 2 | | | |
| 2-Carboxyl-5,7-dihydroxychromen-4-one | 1 | 3 | | 2 | | 5 | | 5 |
| 2-Carboxy-7-hydroxychromen-4- | 5 | 5 | 5 | 5 | 5 | 5 | 5 | 5 |
| one | | | | | | | | |
| Ethyl 5,7-Dihydroxychromen-4- | 1 | 5 | 4 | | 6 | | 7 | |
| one-2-carboxylate | | | | | | | | |
| Zinc Oxide | | | 2 | | | | | |
| UV-Pearl, OMC | 15 | 15 | 15 | 30 | 30 | 30 | 15 | 15 |
| 4-Methylbenzylidene Camphor | | | | 3 | | | | |
| BMDBM | | | | 1 | | | | |
| Phenylbenzimidazole Sulfonic Acid | | | | | 4 | | | |
| Stearyl Alcohol (and) Steareth-7 | 3 | 3 | 3 | 3 | | | | |
| (and) Steareth-10 | | | | | | | | |
| Glyceryl Stearate (and) Ceteth-20 | 3 | 3 | 3 | 3 | | | | |
| Glyceryl Stearate | 3 | 3 | 3 | 3 | | | | |
| Microwax | 1 | 1 | 1 | 1 | | | | |
| Cetearyl Octanoate | 11.5 | 11.5 | 11.5 | 11.5 | | | | |
| Caprylic/Capric Triglyceride | 6 | 6 | 6 | 6 | 14 | 14 | 14 | 14 |
| Oleyl Oleate | 6 | 6 | 6 | 6 | | | | |
| Propylene Glycol | 4 | 4 | 4 | 4 | | | | |
| Glyceryl Stearate SE | | | | | 6 | 6 | 6 | 6 |
| Stearic Acid | | | | | 2 | 2 | 2 | 2 |
| Persea Gratissima | | | | | 8 | 8 | 8 | 8 |
| Propylparaben | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 |
| Tromethamine | | | | | 1.8 | | | |
| Glycerine | | | | | 3 | 3 | 3 | 3 |
| Water | to | to | to | to | to | to | to | to |
| | 100 | 100 | 100 | 100 | 100 | 100 | 100 | 100 |

| | 2-19 | 2-20 | 2-21 | 2-22 | 2-23 | 2-24 | 2-25 | 2-26 | 2-27 | 2-28 |
|---|---|---|---|---|---|---|---|---|---|---|
| Titanium Dioxide | | | | | | | 3 | 3 | | 2 |
| Benzylidene Malonate | 1 | 2 | | | | 1 | 1 | | 1 | 0.5 |
| Polysiloxane | | | | | | | | | | |
| 7,8,3′,4′-Tetrahydroxyflavone | | | | 1 | 2 | | | | 1 | 1 |
| Ethyl 5,7-Dihydroxychromen-4- | 1 | 3 | | 2 | | 5 | | 5 | 2 | |
| on-2-carboxylate | | | | | | | | | | |
| 2-Methyl-5,7-dihydroxy- | 5 | 5 | 5 | 5 | 5 | 5 | 5 | 5 | 5 | 5 |
| chromen-4-one | | | | | | | | | | |
| Methylene Bis-benzotriazolyl | | | 1 | 2 | 1 | | | 1 | 1 | 0.5 |
| Tetramethylbutylphenol | | | | | | | | | | |
| Zinc Oxide | | | | | 5 | 2 | | | | 2 |
| UV-Pearl, OMC | 15 | 15 | 15 | 15 | 15 | 15 | 15 | 15 | 15 | 15 |
| Caprylic/Capric Triglyceride | 14 | 14 | 14 | 14 | 14 | 14 | 14 | 14 | 14 | 14 |
| Oleyl Oleate | | | | | | | | | | |
| Propylene Glycol | | | | | | | | | | |
| Glyceryl Stearate SE | 6 | 6 | 6 | 6 | 6 | 6 | 6 | 6 | 6 | 6 |
| Stearic Acid | 2 | 2 | 2 | 2 | 2 | 2 | 2 | 2 | 2 | 2 |
| Persea Gratissima | 8 | 8 | 8 | 8 | 8 | 8 | 8 | 8 | 8 | 8 |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Glyceryl Stearate. Ceteareth- | | | | | | | | | | |
| 20. Ceteareth-10. Cetearyl | | | | | | | | | | |
| Alcohol. Cetyl Palmitate | | | | | | | | | | |
| Ceteareth-30 | | | | | | | | | | |
| Dicaprylyl Ether | | | | | | | | | | |
| Hexyldecanol, Hexyldexyl | | | | | | | | | | |
| Laurate | | | | | | | | | | |
| Cocoglycerides | | | | | | | | | | |
| Tromethamine | | | | | | | | | | |
| Glycerine | 3 | 3 | 3 | 3 | 3 | 3 | 3 | 3 | 3 | 3 |
| Water | to | to | to | to | to | to | to | to | to | to |
| | 100 | 100 | 100 | 100 | 100 | 100 | 100 | 100 | 100 | 100 |

TABLE 3
Gels, data in % by weight

| | 3-1 | 3-2 | 3-3 | 3-4 | 3-5 | 3-6 | 3-7 | 3-8 | 3-9 | 3-10 |
|---|---|---|---|---|---|---|---|---|---|---|
| a = aqueous gel | | | | | | | | | | |
| Titanium Dioxide | | 2 | 5 | | | | | | | 3 |
| 2-Methyl-5,7-dihydroxy-chromen-4- | | | | 1 | 2 | | | | 1 | 1 |
| one | | | | | | | | | | |
| Ethyl 5,7-Dihydroxychromen-4- | 1 | 3 | | 2 | | 5 | | 5 | 2 | |
| one-2-carboxylate | | | | | | | | | | |
| Benzylidene Malonate Polysiloxane | | | 1 | 1 | 2 | | | | 1 | 1 |
| Methylene Bis-benzotriazolyl | | 1 | | | | 1 | 2 | 1 | | |
| Tetramethylbutylphenol | | | | | | | | | | |
| Zinc Oxide | | | | 2 | | | | 5 | 2 | |
| UV-Pearl, Ethylhexyl | 30 | 15 | 15 | 15 | 15 | 15 | 15 | 15 | 15 | 15 |
| Methoxycinnamate | | | | | | | | | | |
| 4-Methylbenzylidene Camphor | | | | | 2 | | | | | |
| Butylmethoxydibenzoylmethane | | 1 | | | | | | | | |
| Phenylbenzimidazole Sulfonic Acid | | | 4 | | | | | | | |
| Prunus Dulcis | 5 | 5 | 5 | 5 | 5 | 5 | 5 | 5 | 5 | 5 |
| Tocopheryl Acetate | 0.5 | 0.5 | 0.5 | 0.5 | 0.5 | 0.5 | 0.5 | 0.5 | 0.5 | 0.5 |
| Caprylic/Capric Triglyceride | 3 | 3 | 3 | 3 | 3 | 3 | 3 | 3 | 3 | 3 |
| Octyldodecanol | 2 | 2 | 2 | 2 | 2 | 2 | 2 | 2 | 2 | 2 |
| Decyl Oleate | 2 | 2 | 2 | 2 | 2 | 2 | 2 | 2 | 2 | 2 |
| PEG-8 (and) Tocopherol (and) | 0.05 | 0.05 | 0.05 | 0.05 | 0.05 | 0.05 | 0.05 | 0.05 | 0.05 | 0.05 |
| Ascorbyl Palmitate (and) Ascorbic | | | | | | | | | | |
| Acid (and) Citric Acid | | | | | | | | | | |
| Sorbitol | 4 | 4 | 4 | 4 | 4 | 4 | 4 | 4 | 4 | 4 |
| Polyacrylamide (and) C13-14 | 3 | 3 | 3 | 3 | 3 | 3 | 3 | 3 | 3 | 3 |
| Isoparaffin (and) Laureth-7 | | | | | | | | | | |
| Propylparaben | 0.05 | 0.05 | 0.05 | 0.05 | 0.05 | 0.05 | 0.05 | 0.05 | 0.05 | 0.05 |
| Methylparaben | 0.15 | 0.15 | 0.15 | 0.15 | 0.15 | 0.15 | 0.15 | 0.15 | 0.15 | 0.15 |
| Tromethamine | | | 1.8 | | | | | | | |
| Water | to | to | to | to | to | to | to | to | to | to |
| | 100 | 100 | 100 | 100 | 100 | 100 | 100 | 100 | 100 | 100 |

1. Complex compound of the formula I 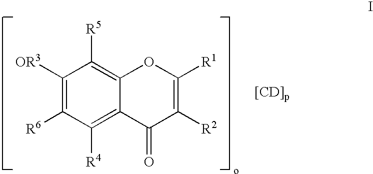
in which
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ- groups, where n=1 to 3,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ stands for H, straight-chain or branched C₁- to C₂₀-alkyl groups, a polyhydroxyl compound, such as, preferably, an ascorbic acid radical or glycosidic radicals, and
R⁸ stands for H or straight-chain or branched C₁- to C₂₀-alkyl groups, where at least 2 of the substituents R¹, R², R⁴—R⁶ are other than H or at least one substituent from R¹ and R² stands for —C(═O)—R⁷ or —C(═O)—OR⁷,
CD stands for a cyclodextrin molecule
o stands for the number 1 and
p stands for a number from the range 0.5 to 3.

2. Complex compound according to claim 1, characterised in that the cyclodextrin CD is an alpha-, beta-, or gamma-cyclodextrin, preferably a gamma-cyclodextrin, which is optionally C₁₋₂₄-alkyl- or C₁₋₂₄-hydroxyalkyl-substituted at one or more hydroxyl groups, particularly preferagbly hydroxypropyl-gamma-cyclodextrin.

3. Complex compound according to claim 1, characterised in that R³ stands for H and R⁴ stands for OH, where at least one of the radicals R⁵ and R⁶ preferably additionally stands for OH.

4. Complex compound according to claim 1, characterised in that characterised in that R⁵ and R⁶ stand for H.

5. Complex compound according to claim 1, characterised in that one of the radicals R¹ or R² stands for H and the other radical stands for —C(═O)—R⁷, —C(═O)—OR⁷ or a straight-chain or branched C₁- to C₂₀-alkyl group.

6. Complex compound according to claim 1, characterised in that the chromone moiety of compound I is a compound selected from the compounds having the formulae IIa-IIn:

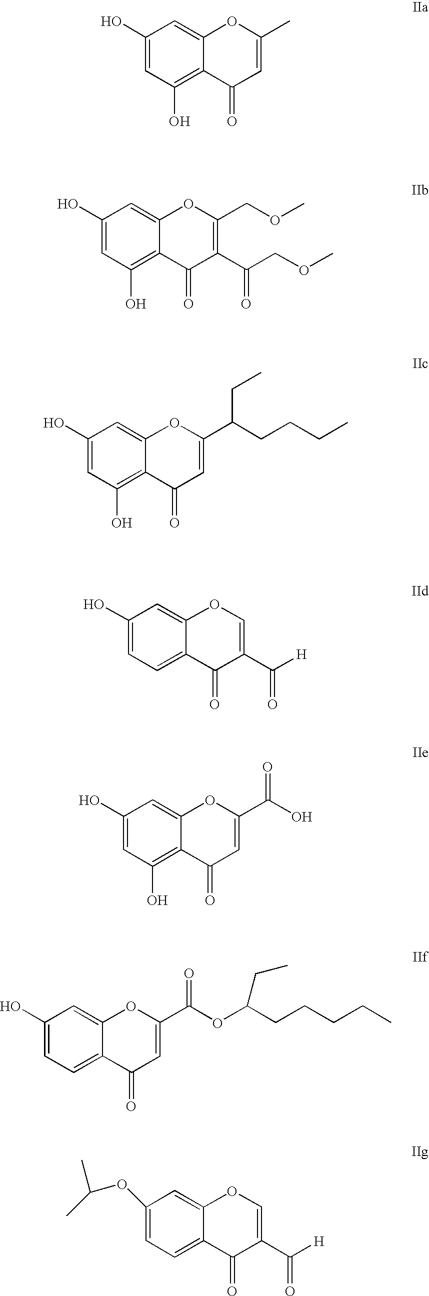

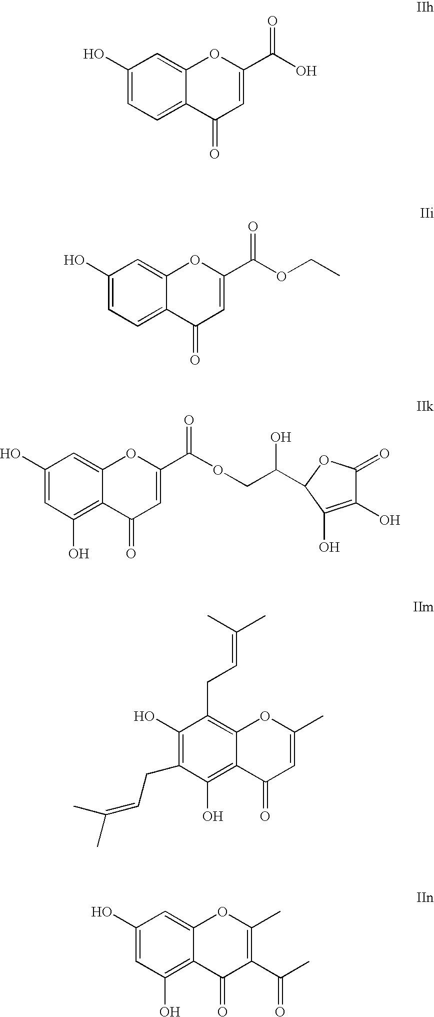

7. Complex compound according to claim 1, characterised in that, in formula I, o is equal to 1 and p is in the range from 1.75 to 2.1, where p is preferably equal to 2.

8. Process for the preparation of complex compounds according to claim 1, characterised in that compounds of the formula II 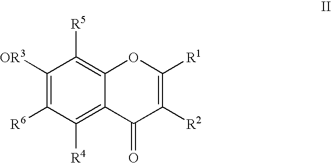
in which
R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups, straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ- groups, where n=1 to 3,
R³ stands for H or straight-chain or branched C₁- to C₂₀-alkyl groups,
R⁴ stands for H or OR⁸,
R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and
R⁷ stands for H, straight-chain or branched C₁- to C₂₀-alkyl groups, a polyhydroxyl compound, such as, preferably, an ascorbic acid radical or glycosidic radicals, and
R⁸ stands for H or straight-chain or branched C₁- to C₂₀-alkyl groups, where at least 2 of the substituents R¹, R², R⁴—R⁶ are other than H or at least one substituent from R¹ and R² stands for —C(═O)—R⁷ or —C(═O)—OR⁷, are reacted with cyclodextrins CD in solution, preferably at elevated temperature.

9. Process according to claim 8, characterised in that cyclodextrin is employed in excess or precisely in the molar ratio 2:1 based on the chromone of the formula II.

10. Composition comprising a suitable vehicle, characterised in that the composition comprises
0.005 to 99% by weight of a complex compound of the formula I according to claim 1 or the composition comprises
0.002 to 70% by weight of cyclodextrin and
0.001 to 60% by weight of at least one compound of the formula II according to claim 8 or topically tolerated salts and/or derivatives thereof.

11. Composition according to claim 10, characterised in that the one or more compounds of the formula I are present in the composition in amounts of 0.01 to 20% by weight, preferably 0.05 to 10% by weight and particularly preferably 0.1 to 5% by weight.

12. Composition according to claim 10, characterised in that the content of cyclodextrins in the composition is 0.01-20.0% by weight, preferably 0.05-10.0% by weight, particularly preferably 0.1-5.0% by weight, in each case based on the total weight of the composition, and the content of compounds of the formula II in the composition is 0.01 to 20% by weight, preferably 0.05 to 10% by weight and particularly preferably 0.1 to 5% by weight, based on the composition as a whole, where the the proportion of the compounds of the formula II in the composition is very especially preferably in the range from 0.1 to 2% by weight, based on the composition as a whole.

13. Composition according to claim 1, characterised in that the composition comprises one or more antioxidants and/or one or more UV filters.

14. Use of a complex compound of the formula I or a composition according to claim 10 for the care, maintenance or improvement of the general state of the skin or hair.

15. Use of a complex compound of the formula I or a composition according to claim 10 for prophylaxis against time- and/or light-induced ageing processes of the human skin or human hair, in particular for prophylaxis against dry skin, wrinkling and/or pigment defects, and/or for the reduction or prevention of damaging effects of UV rays on the skin.

16. Use of a complex compound of the formula I or a composition according to claim 10 for prophylaxis against or reduction of skin unevenness, such as wrinkles, fine lines, rough skin or large-pored skin.

17. Process for the preparation of a composition according to claim 10, characterised in that at least one compound of the formula II and a cyclodextrin or at least one compound of the formula I having radicals as described above is mixed with a cosmetically or dermatologically or food-suitable vehicle.

18. Use of a compound of the formula I for the preparation of composition according to claim 10.
